(19) United States
(12) Patent Application Publication
(10) Pub. No.: US 2022/0260399 A1
(43) Pub. Date: Aug. 18, 2022

(54) VAPORIZER FLOW DETECTION

(71) Applicant: INSTRUMEMS INC., Los Altos, CA (US)

(72) Inventors: Gilad ARWATZ, Mountain View, CA (US); James BORTHWICK, Santa Clara, CA (US); Jeffrey DIAMENT, Santa Cruz, CA (US)

(73) Assignee: INSTRUMEMS INC., Los Altos, CA (US)

(21) Appl. No.: 17/627,442

(22) PCT Filed: Jul. 16, 2020
(86) PCT No.: PCT/US2020/042270

§ 371 Date: Jan. 14, 2022

Related U.S. Application Data
(60) Provisional application No. 62/875,413, filed on Jul. 17, 2019.

(51) Int. Cl. G01F 1/56; G01F 15/04; A24F 40/51; A24F 40/57; A24F 40/85; B08B 7/00; A61M 11/04

(52) U.S. Cl. CPC G01F 1/56; G01F 15/043; A24F 40/51; A24F 40/57; A61M 2205/3368; B08B 7/0071; A61M 11/042; A61M 2205/3334; A61M 2205/3317; A24F 40/85

(57) ABSTRACT

A vaporizer (20) is shaped to define a flow channel (120) that is open to an environment external to the vaporizer at first and second ends of the flow channel. At the first end of the flow channel is a mouthpiece (112) of the vaporizer. A flow sensor (114) includes (a) a nanoscale resistive element (200) disposed at least partially within the flow channel and (b) sensing circuitry (115) configured to measure a change in the nanoscale resistive element due to airflow within the flow channel. Other applications are also described.

### CROSS-REFERENCE TO RELATED APPLICATIONS

The present application claims the benefit of U.S. Provisional Application 62/875,413 to Arwatz et al., entitled “Electronic cigarette flow detection,” filed Jul. 17, 2019, and which is incorporated herein by reference.

### FIELD OF THE INVENTION

The present invention is directed to devices and methods for measuring airflow, and more specifically to measuring airflow in a respiration interface.

### BACKGROUND OF THE INVENTION

Vaporizers, e.g., electronic cigarettes, typically require an activation signal in order to operate, e.g., to enable a heater used for vaporizing a material in order to deliver a substance to a user in the form of an aerosol. The activation signal for the heater may be provided by a switch or button, or through a sensor generally configured to detect a user's inhalation (often referred to as puff detection). Switch-based systems may be considered less desirable due to the requirement for the user to manually switch on and off the activation.

Sensor-based activation systems may use a variety of different sensor types and mechanisms. A common sensor-based method uses one or more pressure sensors to detect a user's puff. For example, a differential pressure sensor may be used to detect a vacuum that is created when a user sucks in air from the mouthpiece of the device. Alternatively, a differential pressure sensor equivalent may be created using two absolute pressure sensors, with one absolute pressure sensor exposed to the vacuum and the other to ambient pressure. In both cases, costs may be high, and the pressure sensors may have decreased reliability, use relatively high power, and/or take up substantial space within the electronic cigarette.

Another sensor type that is used for puff detection is a thermal flow sensor that detects heat transferred from a heater to one or more series of thermocouples (thermopiles) to quantify the amount of flow present. Thermal flow sensors generally use one of two methods. One method uses thermopiles on either side of a heater, whereby the imbalance of heat transfer to the two thermopiles caused by airflow across the sensor is used to measure the amount of airflow present (calorimetric method). The other method uses a pulsed heat signal generated from a heater and detected by thermopiles located a set distance away, whereby the time it takes for the heat signal to move from the heater to the thermopiles indicates the amount of airflow (time-of-flight method). However, due to their overall complexity, these types of sensors also have drawbacks for use in high volume consumer products such as electronic cigarettes.

Other sensors are common in electronic cigarettes as well for purposes beyond puff detection. For example, temperature measurement is often a requirement in order to provide an optimal user experience (e.g., power to the vaporizing heater may be modulated according to the temperature that is measured, such that the electronic cigarette operates at a desired vaporizing temperature). Additionally, temperature measurement is often used for correction of other sensors, including the pressure sensors described above.

Another type of sensor that may be in an electronic cigarette is an accelerometer, which can be used to detect an idle state. For example, if the electronic cigarette has not moved for a period of time (as sensed by the accelerometer) it can be assumed to be idle and go into a sleep mode. Additionally, the accelerometer may be used to capture user inputs by translating sharp motions (e.g., user taps) into commands to provide feedback to the user (e.g., battery charge status), or otherwise alter the mode of operation of the electronic cigarette. These motions can be differentiated to provide different types of functionality (e.g., different number of taps within certain time periods may be translated to different actions performed by the electronic cigarette). There may, however, be difficulty in distinguishing between intentional and unintentional user inputs. Additionally, including another sensor (i.e., other than an airflow sensor such as the airflow sensors described hereinabove) may cost money, use more power, and/or take up extra space within the electronic cigarette.

### SUMMARY OF THE INVENTION

Apparatus and methods are provided for measuring airflow within a vaporizer, e.g., an electronic cigarette, or a vaporizer for the delivery of a therapeutic substance, such as medical cannabis (also known as medical marijuana). The vaporizer is typically shaped to define a flow channel that is open to the environment external to the vaporizer at either end of the flow channel, one end of which is at a mouthpiece of the vaporizer which a user uses to inhale. In accordance with some applications of the present invention, instead of an airflow sensor such as the sensors described in the Background, a microelectromechanical systems (MEMS) flow sensor is used that has (a) a nanoscale resistive element disposed at least partially within the flow channel, and (b) sensing circuitry. Airflow within the flow channel causes a change in the nanoscale resistive element, which is in turn measured by the sensing circuitry, such that the flow sensor is able to measure parameters of the airflow, such as velocity, temperature, and amount.

There is therefore provided, in accordance with some applications of the present invention, apparatus including:

a vaporizer shaped to define a flow channel that is open to an environment external to the vaporizer at first and second ends of the flow channel, the first end of the flow channel being at a mouthpiece of the vaporizer; and a flow sensor including (a) a nanoscale resistive element disposed at least partially within the flow channel and (b) sensing circuitry configured to measure a change in the nanoscale resistive element due to airflow within the flow channel.

For some applications, the vaporizer includes an electronic cigarette.

For some applications, the circuitry is disposed at least in part within the flow channel.

For some applications, the flow sensor includes a flow sensor housing, (a) the nanoscale resistive element and the sensing circuitry being disposed within the flow sensor housing, and (b) the flow sensor housing being disposed at least partially within the flow channel.

For some applications, the flow sensor housing is fully disposed within the flow channel.

For some applications, the flow sensor housing is disposed in a portion of a wall of the flow channel, facing the flow channel.

For some applications, the flow sensor is disposed within a flow sensor housing, and the vaporizer is shaped to define the flow sensor housing.

For some applications, the nanoscale resistive element and the sensing circuitry are disposed at different respective locations within the vaporizer.

For some applications, the sensing circuitry is disposed outside of the flow channel.

For some applications, the vaporizer includes a reservoir configured to hold a material for being vaporized, and the nanoscale resistive element is disposed 5-100 mm away from the reservoir.

For some applications, the nanoscale resistive element is disposed 5-30 mm away from the reservoir.

For some applications:
(a) the vaporizer includes a heater for vaporizing a material within the vaporizer,
(b) the sensing circuitry is configured to measure a velocity of the airflow within the flow channel in response to a measured change in the nanoscale resistive element, and
(c) the vaporizer is configured to activate the heater when the measured velocity of the airflow within the flow channel reaches a threshold value.

For some applications:
(a) the sensing circuitry includes switching circuitry, configured to switch the sensing circuitry between:
   (i) measuring velocity of the airflow within the flow channel in response to a measured change in the nanoscale resistive element, and
   (ii) measuring temperature within the flow channel in response to a measured change in the nanoscale resistive element,
(b) the flow sensor is configured to determine a temperature-compensated velocity value of the airflow within the flow channel by using the measured temperature to correct the measured velocity of the airflow, and
(c) the vaporizer is configured to activate the heater when the temperature-compensated velocity value reaches a threshold value.

For some applications, the switching circuitry is configured to switch the sensing circuitry between (i) operating the nanoscale resistive element in a Constant Temperature Anemometry (CTA) mode of operation, in which the sensing circuitry measures velocity of the airflow within the flow channel in response to a measured change in the nanoscale resistive element, and (ii) operating the nanoscale resistive element in a Constant Current Anemometry (CCA) mode of operation, in which the sensing circuitry measures temperature within the flow channel in response to a measured change in the nanoscale resistive element.

For some applications, the switching circuitry is configured to switch the sensing circuitry between (i) operating the nanoscale resistive element in a Constant Temperature Anemometry (CTA) mode of operation, in which the sensing circuitry measures velocity of the airflow within the flow channel in response to a measured change in the nanoscale resistive element, and (ii) operating the nanoscale resistive element in a Constant Voltage Anemometry (CVA) mode of operation, in which the sensing circuitry measures temperature within the flow channel in response to a measured change in the nanoscale resistive element.

For some applications, the sensing circuitry is configured to operate the nanoscale resistive element in a Constant Current Anemometry (CCA) mode of operation, and the switching circuitry is configured to switch the sensing circuitry between (i) operating the nanoscale resistive element at a first current level such that the sensing circuitry measures velocity of the airflow within the flow channel in response to a measured change in the nanoscale resistive element and (ii) operating the nanoscale resistive element at a second current level that is lower than the first current level such that the sensing circuitry measures temperature within the flow channel in response to a measured change in the nanoscale resistive element.

For some applications, the sensing circuitry is configured to operate the nanoscale resistive element in a Constant Voltage Anemometry (CVA) mode of operation, and the switching circuitry is configured to switch the sensing circuitry between (i) operating the nanoscale resistive element at a first voltage level such that the sensing circuitry measures velocity of the airflow within the flow channel in response to a measured change in the nanoscale resistive element and (ii) operating the nanoscale resistive element at a second voltage level that is lower than the first voltage level such that the sensing circuitry measures temperature within the flow channel in response to a measured change in the nanoscale resistive element.

For some applications:
(a) the nanoscale resistive element is a first nanoscale resistive element,
(b) the flow sensor further includes a second nanoscale resistive element,
(c) the sensing circuitry is configured to operate the first and second nanoscale resistive elements, such that:
   (i) the sensing circuitry measures velocity of the airflow within the flow channel in response to a measured change in the first nanoscale resistive element, and
   (ii) the sensing circuitry measures temperature within the flow channel in response to a measured change in the second nanoscale resistive element,
(d) the flow sensor is configured to determine a temperature-compensated velocity value of the airflow within the flow channel by using the measured temperature to correct the measured velocity of the airflow, and
(e) the vaporizer is configured to activate the heater when the temperature-compensated velocity value reaches a threshold value.

For some applications, the sensing circuitry is configured to:
operate the first nanoscale resistive element in a Constant Temperature Anemometry (CTA) mode of operation, in which the sensing circuitry measures velocity of the airflow within the flow channel in response to a measured change in the first nanoscale resistive element, and
operate the second nanoscale resistive element in a Constant Current Anemometry (CCA) mode of operation, in which the sensing circuitry measures temperature within the flow channel in response to a measured change in the second nanoscale resistive element.

For some applications, the sensing circuitry is configured to:
operate the first nanoscale resistive element in a Constant Temperature Anemometry (CTA) mode of operation, in which the sensing circuitry measures velocity of the airflow within the flow channel in response to a measured change in the first nanoscale resistive element, and
operate the second nanoscale resistive element in a Constant Voltage Anemometry (CVA) mode of operation, in which the sensing circuitry measures temperature within the flow channel in response to a measured change in the second nanoscale resistive element.

For some applications, the sensing circuitry is configured to operate the first and second nanoscale resistive elements in a Constant Current Anemometry (CCA) mode, the first nanoscale resistive element being operated at a first current level, and the second nanoscale resistive element being operated at a second current level that is lower than the first current level.

For some applications, the sensing circuitry is configured to operate the first and second nanoscale resistive elements in a Constant Voltage Anemometry (CVA) mode, the first nanoscale resistive element being operated at a first voltage level, and the second nanoscale resistive element being operated at a second voltage level that is lower than the first voltage level.

For some applications the flow sensor is configured to perform a cleaning cycle, the sensing circuitry being configured to increase the temperature of the nanoscale resistive element during the cleaning cycle.

For some applications, the sensing circuitry is configured to increase the temperature of the nanoscale resistive element to 300-1000 degrees Celsius during the cleaning cycle.

For some applications, the flow sensor is configured to perform the cleaning cycle at fixed time intervals.

For some applications, the flow sensor is configured to automatically perform the cleaning cycle when the vaporizer is connected to a power source external to the vaporizer.

For some applications:
(A) the sensing circuitry is configured to operate the nanoscale resistive element in a low-power sensing mode in which the sensing circuitry is configured to:
   (i) apply electrical energy to the nanoscale resistive element,
   (ii) detect a change in an electrical property associated with the application of the electrical energy to the nanoscale resistive element, the extent of the change in the electrical property being due to the extent of loss of thermal energy from the nanoscale resistive element, and
   (iii) identify that the nanoscale resistive element is in a fouled state based on the detected change in the electrical property, and
(B) the flow sensor is configured to perform the cleaning cycle in response to the sensing circuitry determining that the nanoscale resistive element is in the fouled state.

For some applications, during the cleaning cycle the sensing circuitry is configured to operate the nanoscale resistive element at a burn-off power level that is 50-150 times higher than a sensing power level at which the sensing circuitry operates the nanoscale resistive element during the low-power sensing mode.

For some applications, the sensing circuitry is configured to, during the low-power sensing mode:
(i) apply the electrical energy to the nanoscale resistive element to increase the temperature of the nanoscale resistive element, the increase in temperature of the nanoscale resistive element inducing an increase in resistance of the nanoscale resistive element,
(ii) detect the increase in resistance of the nanoscale resistive element that is due to the increase in temperature of the nanoscale resistive element, the extent of the increase in temperature of the nanoscale resistive element being due to the extent of loss of thermal energy from the nanoscale resistive element, and
(iii) identify that the nanoscale resistive element is in the fouled state based on the detected increase in resistance of the nanoscale resistive element.

For some applications, the sensing circuitry is configured to, during the low-power sensing mode:
(i) apply the electrical energy to the nanoscale resistive element by regulating a current applied to the nanoscale resistive element, and
(ii) detect the increase in resistance of the nanoscale resistive element by monitoring a voltage across the nanoscale resistive element in response to regulating the current.

For some applications, the sensing circuitry is configured to, during the low-power sensing mode:
(i) apply the electrical energy to the nanoscale resistive element by regulating a voltage applied across the nanoscale resistive element, and
(ii) detect the increase in resistance of the nanoscale resistive element by monitoring a current in the nanoscale resistive element in response to regulating the voltage.

For some applications, the sensing circuitry is configured to, during the low-power sensing mode:
(i) apply the electrical energy to the nanoscale resistive element to increase the temperature of the nanoscale resistive element by beginning to apply the electrical energy while the resistance of the nanoscale resistive element is at a baseline resistance value, the increase in temperature of the nanoscale resistive element inducing the resistance of the nanoscale resistive element to increase to a resistance value that is above the baseline resistance value,
(ii) detect an extent of the increase in the resistance of the nanoscale resistive element from the baseline resistance value, and
(iii) identify that the nanoscale resistive element is in the fouled state in response to the resistance of the nanoscale resistive element increasing to a resistance value that is less than a threshold value above the baseline resistance value.

For some applications, the sensing circuitry is configured to determine whether the resistance of the nanoscale resistive element has increased to the threshold value at a time t while the temperature of the nanoscale resistive element is still rising due to the applied electrical energy.

For some applications, time t is 0.1-10 milliseconds after the start of the application of the electrical energy to the nanoscale resistive element.

For some applications:
(a) the threshold value is a first threshold value, and
(b) the sensing circuitry is configured to identify that the nanoscale resistive element is in a severely-fouled state in response to the resistance of the nanoscale resistive element increasing to a resistance value at time t that is less than a second threshold value above the baseline resistance value, the second threshold value being lower than the first threshold value.

For some applications, the vaporizer is configured to generate an alert in response to the identification that the nanoscale resistive element is in the severely-fouled state, the alert prompting a user of the vaporizer to blow into the flow channel.

For some applications:

subsequently to the user blowing into the flow channel, but prior to a new activation of the heater, the sensing circuitry is configured to, while operating the nanoscale resistive element in the low-power sensing mode, identify that the nanoscale resistive element is in a fouled state in response to the resistance of the nanoscale resistive element increasing to a resistance value that is above (I) the second threshold value above the baseline resistance value and below (II) the first threshold value above the baseline resistance value, and the flow sensor is configured to activate the cleaning cycle in response to the sensing circuitry determining that the nanoscale resistive element is in the fouled state.

For some applications, the vaporizer is configured to generate an alert selected from the group consisting of: a visual alert, an audible alert, and a tactile alert.

For some applications, the vaporizer is configured to wirelessly connect to an external device, and the vaporizer is configured to generate the alert via the external device.

For some applications, the sensing circuitry is configured to, during the low-power sensing mode:
(i) regulate the temperature of the nanoscale resistive element by applying the electrical energy to the nanoscale resistive element,
(ii) detect a change in a level of power input to the nanoscale resistive element in order to regulate the temperature of the nanoscale resistive element, the extent of the change in power input being due to the extent of loss of thermal energy from the nanoscale resistive element, and
(iii) identify that the nanoscale resistive element is in the fouled state based on the change in the level of power input to the nanoscale resistive element.

For some applications:
(A) during the cleaning cycle, the sensing circuitry is configured to:
   (i) increase the temperature of the nanoscale resistive element by applying a voltage across the nanoscale resistive element, and
   (ii) monitor a resistance of the nanoscale resistive element in response to the applied voltage, and
(B) the flow sensor is configured to terminate the cleaning cycle when the resistance of the nanoscale resistive element passes a clean-state threshold value.

For some applications, the sensing circuitry is configured to increase the temperature of the nanoscale resistive element by applying a voltage of 1-2 V to the nanoscale resistive element.

For some applications, the sensing circuitry is configured to increase the temperature of the nanoscale resistive element by applying a voltage that is 50-150 times higher than a sensing voltage applied to the nanoscale resistive element in order to measure temperature within the flow channel.

For some applications:
(A) during the cleaning cycle, the sensing circuitry is configured to:
   (i) increase the temperature of the nanoscale resistive element by applying a voltage across the nanoscale resistive element, and
   (ii) monitor the temperature of the nanoscale resistive element in response to the applied voltage, and
(B) the flow sensor is configured to terminate the cleaning cycle when the temperature of the nanoscale resistive element passes a clean-state threshold value.

For some applications, the sensing circuitry is configured to increase the temperature of the nanoscale resistive element by applying a voltage of 1-2 V to the nanoscale resistive element.

For some applications, the sensing circuitry is configured to increase the temperature of the nanoscale resistive element by applying a voltage that is 50-150 times higher than a sensing voltage that is applied to the nanoscale resistive element in order to measure temperature within the flow channel.

For some applications, the flow sensor is configured to determine an amount of material vaporized within the vaporizer in response to the measured velocity of the airflow within the flow channel subsequent to the activation of the heater.

For some applications, the vaporizer includes a processor configured to regulate the amount of material vaporized in response to the measured velocity of the airflow within the flow channel subsequent to the activation of the heater.

For some applications, the processor is configured to regulate the amount of material vaporized in a given puff of a user.

For some applications, the processor is configured to regulate the amount of material vaporized per day.

For some applications, the processor is configured to receive an input of a maximum daily amount of material to be vaporized and to deactivate the heater when the maximum daily amount of material to be vaporized has been vaporized.

For some applications, the processor is configured to assist a user to decrease the user's dependence on an addictive drug, the addictive drug being the vaporized material, by running an algorithm that gradually decreases the maximum daily amount of material to be vaporized.

For some applications, the processor is configured to receive an input of a maximum per-puff amount of material to be vaporized and, for each puff of a user, to deactivate the heater when the maximum per-puff amount of material to be vaporized has been vaporized.

For some applications, the processor is configured to run an algorithm that gradually decreases the maximum per-puff amount of material to be vaporized so as to assist the user to decrease the user's dependence on an addictive drug, the addictive drug being the vaporized material.

For some applications, the sensing circuitry is configured to:
(a) operate the nanoscale resistive element in a low-power Constant Temperature Anemometry (CTA) puff detection mode, in which the nanoscale resistive element is maintained at a constant differential temperature relative to ambient temperature, and
(b) when a puff is detected, switch to operating the nanoscale resistive element in a high-power CTA puff characterization mode, in which the nanoscale resistive element is maintained at a constant absolute temperature.

For some applications, the sensing circuitry is configured to:
(a) operate the nanoscale resistive element in a low-power Constant Temperature Anemometry (CTA) puff detection mode, in which the sensing circuitry intermittently heats the nanoscale resistive element in order to measure the velocity of the airflow within the flow channel, and
(b) when a puff is detected, switch to a high-power CTA puff characterization mode, in which the nanoscale resistive element is maintained at a constant absolute temperature.

For some applications, the vaporizer include an obstruction disposed within the flow channel configured to cause vortex shedding in response to airflow moving past the obstruction, and the flow sensor is configured to sense the vortex shedding and, in response to the sensed vortex shedding, determine a direction of the airflow within the flow channel.

For some applications:

the flow sensor includes a plurality of nanoscale resistive elements disposed at a plurality of respective angles with respect to each other, the sensing circuitry is configured to operate each one of the plurality of nanoscale resistive elements, and to generate a respective sensing signal for each nanoscale resistive element, and the flow sensor is configured to determine a direction of one-dimensional airflow within the flow channel in response to the respective sensing signals from each of the plurality of nanoscale resistive elements.

For some applications, the flow sensor is configured to determine a direction of the airflow within the flow channel in response to the sensing circuitry measuring a change in temperature of the nanoscale resistive element due to airflow within the flow channel.

For some applications:

the sensing circuitry is configured to generate a sensor signal indicative of the measured velocity of the airflow within the flow channel, the vaporizer includes a processor configured to analyze a level of fluctuations in the sensor signal, and the processor is configured to determine a direction of the airflow within the flow channel in response to the level of fluctuations in the sensor signal.

For some applications:
the vaporizer includes a physical structure positioned to affect airflow at the nanoscale resistive element, the sensing circuitry is configured to generate a sensor signal indicative of the measured velocity of the airflow within the flow channel, and the vaporizer includes a processor is configured to determine a direction of the airflow within the flow channel in response to the sensor signal.

For some applications, the physical structure is positioned along the flow channel between the nanoscale resistive element and the mouthpiece of the vaporizer.

For some applications, the physical structure is positioned along the flow channel between the nanoscale resistive element and a distal end of the flow channel.

For some applications, the processor is configured to determine the direction of the airflow within the flow channel in response to a level of fluctuation in the sensor signal, by identifying (i) a high level of fluctuation in the sensor signal as being indicative of airflow in a first direction with respect to the physical structure and (ii) a low level of fluctuation in the sensor signal as being indicative of airflow in a second direction with respect to the physical structure, opposite the first direction.

For some applications, the physical structure is positioned along the flow channel between the nanoscale resistive element and the mouthpiece of the vaporizer, and the processor is configured to identify (i) airflow within the flow channel in the first direction as a blow, and (ii) airflow within the flow channel in the second direction as a puff.

For some applications, the vaporizer is configured to (i) activate the heater when the measured velocity of airflow within the flow channel during a puff reaches the threshold value, and (ii) not activate the heater when the processor identifies the airflow within the flow channel as a blow.

For some applications, the flow sensor is configured to determine an amount of material vaporized within the vaporizer in response to the measured velocity of the airflow within the flow channel subsequent to the activation of the heater.

For some applications, the physical structure is positioned along the flow channel between the nanoscale resistive element and a distal end of the flow channel, and the processor is configured to identify (i) airflow within the flow channel in the first direction as a puff, and (ii) airflow within the flow channel in the second direction as a blow.

For some applications, the vaporizer is configured to (i) activate the heater when the measured velocity of airflow within the flow channel during a puff reaches the threshold value, and (ii) not activate the heater when the processor identifies the airflow within the flow channel as a blow.

For some applications, the flow sensor is configured to determine an amount of material vaporized within the vaporizer in response to the measured velocity of the airflow within the flow channel subsequent to the activation of the heater.

For some applications:

the sensing circuitry is configured to generate a sensor signal indicative of the measured velocity of the airflow within the flow channel; and

the vaporizer includes a processor configured to analyze the sensor signal and determine a differential pressure within the flow channel.

For some applications:

the flow sensor is a first flow sensor and the nanoscale resistive element is a first nanoscale resistive element, the apparatus further includes at least a second flow sensor including (a) a second nanoscale resistive element disposed at least partially within the flow channel, the second flow sensor and (b) sensing circuitry configured to measure a change in the second nanoscale resistive element due to airflow within the flow channel, and the first and second flow sensors are configured to determine a direction of airflow within the flow channel in response to determining which of the first and second flow sensors measures a change in its nanoscale resistive element prior to the other one of the first and second flow sensors measuring the same change in its nanoscale resistive element.

For some applications, the vaporizer includes a one-way valve disposed within the flow channel and positioned so as to block the nanoscale resistive element from being exposed to airflow in a first direction, such that any substantial airflow sensed by the flow sensor is airflow in a second direction opposite the first direction.

For some applications, the sensing circuitry is configured to operate the nanoscale resistive element with Constant Current Anemometry (CCA).

For some applications, the sensing circuitry is configured to operate the nanoscale resistive element with Constant Temperature Anemometry (CTA).

For some applications, the sensing circuitry is configured to operate the nanoscale resistive element with Constant Voltage Anemometry (CVA).

There is further provided, in accordance with some applications of the present invention, a method including:

using a flow sensor disposed within the flow channel of a vaporizer, (i) the vaporizer being shaped to define a flow channel that is open to an environment external to the vaporizer at first and second ends of the flow channel, the first flow channel being at a mouthpiece of the vaporizer, and (ii) the flow sensor including at least one nanoscale resistive element disposed at least partially within the flow channel:

measuring a velocity of the airflow within the flow channel by measuring a change in the at least one nanoscale resistive element due to airflow within the flow channel; and

activating a heater for vaporizing a material within the vaporizer when the measured velocity reaches a threshold value.

For some applications, the method further includes:

measuring a temperature within the flow channel by measuring a change in a nanoscale resistive element selected from the group consisting of: the at least one nanoscale resistive element, and another nanoscale resistive element;

determining a temperature-compensated velocity value by using the measurement of the temperature within the flow channel to correct the measured velocity of the airflow; and

activating the heater when the temperature-compensated velocity value reaches a threshold value.

The present invention will be more fully understood from the following detailed description of applications thereof, taken together with the drawings, in which:

### BRIEF DESCRIPTION OF DRAWINGS

FIG. 1 is a schematic illustration of an electronic cigarette, in accordance with some applications of the present invention;

FIGS. 2A-C are schematic illustrations of three configurations of a flow sensor illustrated in wafer die form, including support and connectivity components, in accordance with some applications of the present invention;

FIG. 3 is a schematic illustration of a nanoscale film resistive element on a substrate, used as a flow sensing nanoscale resistive element, illustrated in wafer die form, including support and connectivity components, in accordance with some applications of the present invention;

FIG. 4A is a block diagram depicting electronics relating to airflow detection and activation for an exemplary electronic cigarette, in accordance with some applications of the present invention;

FIGS. 4B-H are graphs showing various measurement signals from the flow sensor, in accordance with some applications of the present invention;

FIG. 5A illustrates electronics of a nanoscale wire or film resistive element based thermal flow sensor in block diagram form, in accordance with some applications of the present invention;

FIG. 5B is a block diagram depicting a specific implementation of the electronics shown in FIG. 5A, in accordance with some applications of the present invention;

FIG. 6 is a flow chart illustrating a method of using a nanoscale wire or film resistive element based thermal flow sensor for measuring airflow and temperature, in accordance with some applications of the present invention;

FIG. 7 is a flow chart illustrating a method of using a nanoscale wire or film resistive element based thermal flow sensor for measuring airflow and temperature using a single nanoscale wire or film resistive element, in accordance with some applications of the present invention;

FIG. 8 is a schematic illustration showing a flow channel of an electronic cigarette with an exemplary nanoscale flow sensor placed in the flow channel, in accordance with some applications of the present invention;

FIG. 9 is a data graph showing two data curves depicting flowrate versus pressure within the flow channel in an experiment carried out by the inventors, in accordance with some applications of the present invention;

FIG. 10 is a flow chart depicting closed loop sensing for sensor fouling, including detecting a severely-fouled state of the nanoscale resistive element and cleaning the nanoscale resistive element, in accordance with some applications of the present invention; and

FIG. 11 is a data graph showing a baseline resistance curve of the nanoscale resistive element when it is clean, and data curves showing the resistance of the nanoscale resistive element during a cleaning cycle, starting from various different degrees of fouling, in accordance with some applications of the present invention.

### DETAILED DESCRIPTION OF THE INVENTION

Provided herein are devices and methods for measuring airflow in a respiration interface using a microelectromechanical systems (MEMS) thermal flow sensor, in accordance with some applications of the present invention. The scope of the present invention includes any respiration interface which conventionally measures airflow, and/or in which it would be beneficial to measure airflow, e.g., recreational devices such as electronic cigarettes, therapeutic devices, medical devices (e.g., nebulizers or vaporizers used for the delivery of a medical substance, ventilators, anesthesia machines, metered dose inhalers, dry powder inhalers), and sports training devices. Embodiments of the present invention may also include any device in which it would be beneficial to measure airflow, e.g., devices used in manufacturing processes, industrial applications, vehicles, aircraft, e.g., drones, HVAC (heating, ventilation, and air conditioning) in buildings, flow sensing in fire detection systems, and consumer airflow devices (e.g., vacuum cleaners and hair dryers).

Hereinbelow is provided a detailed example within the scope of the present invention, i.e., devices and methods for measuring airflow in a vaporizer 20, e.g., an electronic cigarette 100, or a vaporizer for the delivery of a therapeutic substance, such as medical cannabis, using a microelectromechanical systems (MEMS) thermal flow sensor, in accordance with some applications of the present invention. The airflow to be detected and measured is generated by a user inhaling on a mouthpiece of vaporizer 20 (referred to throughout the present application, including in the claims, as a “puff”), or exhaling into the mouthpiece of the vaporizer (referred to throughout the present application, including in the claims, as a “blow”). The airflow is measured, and according to the measurement, for example, vaporizer 20 will or will not activate a heater for vaporizing a material within the electronic cigarette. Typically, heater 106 operates by conduction (i.e., in direct contact with material to be vaporized). For some applications, e.g., when the material to be vaporized is a liquid, heater 106 typically includes a heating element that is a coil of resistive wire surrounding a wick.

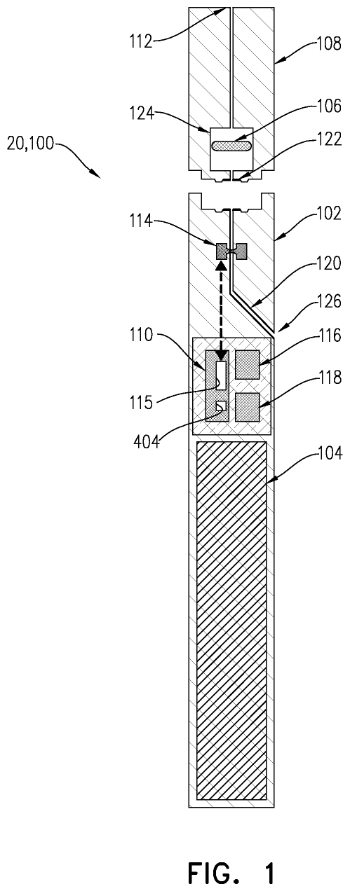

Reference is now made to FIG. 1, which is a schematic illustration of vaporizer 20, in accordance with some applications of the present invention. Vaporizer 20 may be, for example, an electronic cigarette 100 or a vaporizer for the delivery of a therapeutic substance. A vaporizer housing 102, which houses various components of vaporizer 20, may be provided in a variety of shapes, although an elongated shape is common for ease of use and familiarity (e.g., with respect to a standard cigarette). Vaporizer housing 102 may include a battery 104, which is generally a rechargeable battery (e.g., a lithium-ion battery), a heater 106 for heating the material to be vaporized, a reservoir 108 for holding the material to be vaporized, a vaporization chamber 124 where the material is vaporized, a controller printed circuit board (PCB) 110, a mouthpiece 112 which the user inhales through, a flow channel 120 that is used to convey ambient air through the vaporization chamber during inhalation, a flow sensor 114 for detecting airflow and activating the material vaporization, an accelerometer 116 for detecting movement of the device, a capacitive touch sensor 118 for detecting when a user touches the device, and/or an airtight or partially airtight seal 122 positioned where the airflow channel in the device interfaces with the airflow channel of an optional detachable cartridge. For some applications, reservoir 108 is disposed in a detachable cartridge or pod.

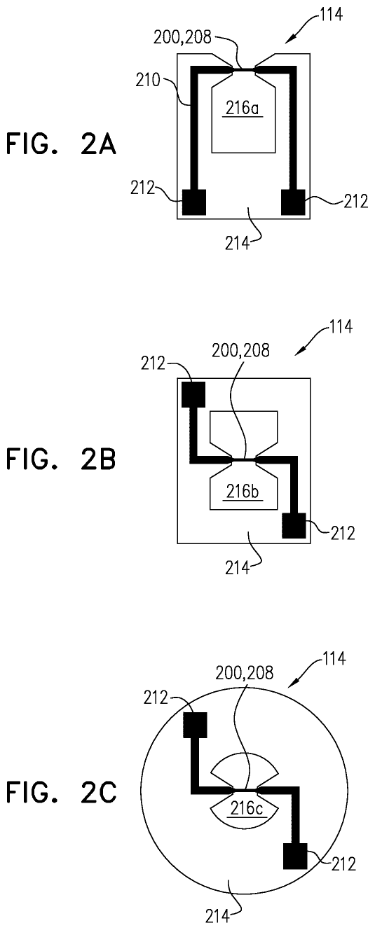

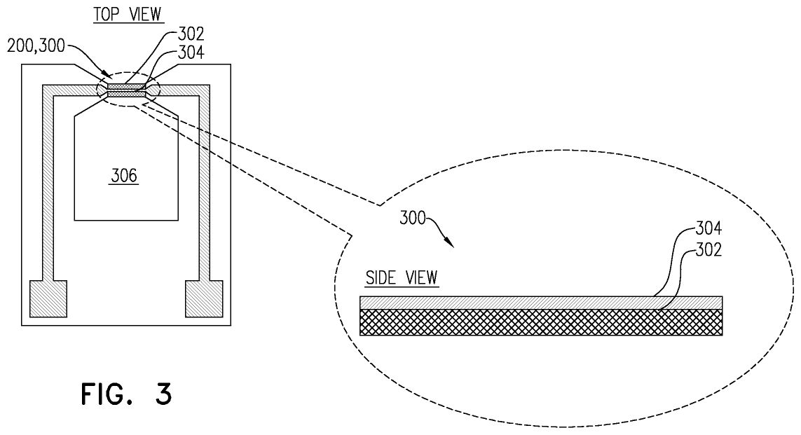

For some applications, flow sensor 114 is a MEMS-based thermal sensor for sensing airflow within flow channel 120 that includes (A) a nanoscale resistive element 200 (FIG. 2), such as a nanoscale wire 208, also referred to herein as nanowire 208 (such as is shown in FIGS. 2A-C), or nanoscale film resistive element 300 (such as is shown in FIG. 3), and (B) an electronic circuit, referred to hereinbelow as sensing circuitry 115, configured to measure a change in the nanoscale resistive element due to airflow within the flow channel. For measuring airflow, nanoscale resistive element 200 is typically heated by sensing circuitry 115, which provides power to nanoscale resistive element 200 in a controlled manner. Heat is then removed from the nanoscale resistive element by the airflow, and a resulting change in the nanoscale resistive element is detected and used to quantify the air velocity, and from that, optionally, the amount of airflow. For some applications, sensing circuitry 115 may be located on controller PCB 110. Alternatively, for some applications, sensing circuitry 115 may be located on the same substrate as nanoscale resistive element 200 itself, further describe hereinbelow.

Nanoscale resistive element 200 is typically made of a material that has a non-zero Thermal Coefficient of Resistance (TCR). For example, nanowire 208 or nanoscale film resistive element 300 may comprise platinum. Other metals or compositions may be used as well. Nanoscale resistive element 200 undergoes changes in resistance due to heat generated within it (i.e., from Joule heating) and due to the heat that is generated within it being removed by the airflow; these changes in resistance are utilized to quantify airflow surrounding nanoscale resistive element 200.

For quantifying the amount of airflow, nanoscale resistive element 200 is typically connected to a Constant Current Anemometry (CCA) circuit, Constant Temperature Anemometry (CTA) circuit, or Constant Voltage Anemometry (CVA) circuit. A CCA circuit operates by providing a near constant current to nanoscale resistive element 200 and monitoring the change in resistance in nanoscale resistive element 200 in order to quantify the amount of airflow. A CTA circuit operates by maintaining nanoscale resistive element 200 near a constant, elevated temperature and monitoring the change in power required to maintain constant temperature in order to quantify the amount of airflow. A CVA circuit operates by providing a near constant voltage across nanoscale resistive element 200 and monitoring the change in resistance in nanoscale resistive element 200 in order to quantify the amount of airflow.

Reference is now made to FIGS. 2A-C, which are schematic illustrations of three configurations of flow sensor 114 illustrated in wafer die form, including support and connectivity components, in accordance with some applications of the present invention. Flow sensor 114 is typically disposed at least partially within flow channel 120, such that at least nanoscale resistive element 200 is exposed to airflow within flow channel 120. FIG. 2A shows a configuration of flow sensor 114 that may reduce physical obstruction of flow channel 120. The conductive free-standing (where the term “free-standing” refers to the nanowire not having any support structure other than connections at each end of the nanowire) nanowire 208 is fully exposed to the flow, and conductive prongs 210 connect the two ends of nanowire 208 to respective solder pads 212. The conductive material that comprises nanowire 208 can be the same material or a different material than that which comprises conductive prongs 210 and solder pads 212. The conductive materials are typically layered on top of a nonconductive substrate 214 that has an opening 216a surrounding nanowire 208 to allow for minimal flow obstruction. FIGS. 2B and 2C show configurations of flow sensor 114 that can be interfaced with rectangular and circular flow paths, respectively. As such, opening 216b in FIG. 2B is generally rectangular and opening 216c in FIG. 2C is generally circular.

For some applications, nanowire 208 has one or more of the following dimensions:
a longitudinal length of at least 30 microns and/or less than 250 microns, e.g., at least 60 microns and/or less than 100 microns, and
a typically, but not necessarily, rectangular cross-section having a width of at least 1 micron and/or less than 2 microns and/or a height of at least 0.1 microns and/or less than 0.2 microns, the cross-section being taken perpendicular to a direction of current flow in the nanoscale resistive element.

Reference is now made to FIG. 3, which is a schematic illustration of nanoscale film resistive element 300 on a substrate, used as a flow sensing nanoscale resistive element 200, illustrated in wafer die form, including support and connectivity components, in accordance with some applications of the present invention. The top view of nanoscale film resistive element 300 shows a non-conductive supportive substrate 302 in close proximity to a resistive film 304. Supportive substrate 302 can be the same material as the bulk supportive substrate 214 shown in FIG. 2, or it can be a different material. In some applications, there is a partial opening 306 to allow for minimal obstruction of the flow. Alternatively, supportive substrate 302 may be expanded to fill the entire space between the support structure, i.e., there may be no opening 306. The side view of nanoscale film resistive element 300 shows how resistive film 304 is layered on top of substrate 302. In some applications, resistive film 304 is layered directly on top of the substrate 302. Alternatively, there may be an adhesive layer between the resistive film 304 and substrate 302 to facilitate the deposition of the resistive film 304 on substrate 302. In FIG. 3, supportive substrate 302 is shown as having a rectangular cross section by way of example and not limitation, i.e., supportive substrate may have various different shapes. In some applications, substrate 302 is tapered, or the density of the material of substrate 302 is decreased by means of holes or slots in order to minimize the thermal mass adjacent to resistive film 304, while still providing adequate support.

In some embodiments, the nanoscale film resistive element 300 has one or more of the following dimensions:
a longitudinal length of at least 30 microns and/or less than 250 microns, e.g., at least 60 microns and/or less than 100 microns,
a typically, but not necessarily, rectangular cross-section having a width of at least 1 micron and/or less than 2 microns, and/or a height of at least 0.1 microns and/or less than 0.2 microns, the cross-section being taken perpendicular to a direction of current flow in the resistive film.

As described hereinabove, nanoscale resistive element 200 may be of any suitable material with a non-zero TCR, and a material commonly chosen is platinum with a positive TCR of 2000-3920 ppm/° C., depending on purity, annealing, and other manufacturing steps. Other materials such as polysilicon may be used, e.g., where accuracy is not as much of a priority as cost. Additionally, in some applications, a single nanoscale resistive element 200 is included per die, while in other applications, multiple nanoscale resistive elements are included per die.

In general, materials and processes used in the MEMS-based thermal flow sensors described hereinabove lend themselves to low cost in comparison to other sensor types and technologies. Nanoscale resistive element 200 and its support structure are compatible with standard semiconductor manufacturing processes, which can be leveraged for high-volume production with the ability to scale costs to low levels, e.g., as volumes increase into the hundreds of millions of units. Furthermore, semiconductor manufacturing requires tightly-controlled processes, which result in a very consistent finished product requiring little or no characterization of each individual unit.

Other cost savings can be achieved by combining nanoscale resistive element 200 onto the same substrate as the electronics used for sensing circuitry 115. In some embodiments, a monolithic solution is created including one or more nanoscale resistive element 200 on a silicon substrate that also includes electronics (op-amps, instrumentation amplifiers, ADCs, communication logic, etc.) as an Application-Specific Integrated Circuit (ASIC). Combining both the MEMS portion of the sensor with the supporting electronics can lower costs relative to other sensor types (e.g., pressure sensors) that require a packaging step to combine the sensing element and the electronics.

In addition to lower costs provided by MEMS-based thermal flow sensors relative to conventional sensing solutions for vaporizers (e.g., pressure sensors), the MEMS-based thermal flow sensors provided herein also enhance robustness and reliability. Pressure sensors are common points of failure in vaporizers, which generally do not perform well in the environment of a vaporizer, e.g., an electronic cigarette. The pressure sensors typically operate by quantifying the deflection of a fine membrane due to pressure fluctuations. However, the inside of a vaporizer, e.g., an electronic cigarette, may present many challenges to this fine membrane. Over time, particulates, aerosols, and liquids may come into contact with the membrane—saturating or damaging the surface and ultimately degrading the sensor performance. In order to mitigate these contamination effects, the pressure sensors are often isolated from large particles via placement in a small channel or tube. However, this presents the possibility for another failure mode: complete or partial blockage of the small channel or tube, which again may lead to inaccurate sensor measurements. In contrast, the simple and static geometry of the nanoscale wire 208 or nanoscale film resistive element 300 described herein provides a more robust solution to the flexible membrane of a pressure sensor.

Reference is again made to FIG. 1. Typically, flow sensor 114 is mounted inside vaporizer housing 102 such that nanonoscale wire 208 or nanoscale film resistive element 300 is sufficiently isolated from reservoir 108, in order to minimize unwanted fouling of nanoscale wire 208 or nanoscale film resistive element 300. For example, heater 106 may be positioned between flow sensor 114 and reservoir 108 and therefore acts as a barrier to any liquid leaving the reservoir and and migrating toward nanoscale resistive element 200. For some applications, nanoscale resistive element 200 may be sufficiently isolated from reservoir 108 by being disposed within flow channel 120 such that there is some distance between flow sensor 114 and reservoir 108, e.g., flow sensor 114 may be disposed at least 5 mm and/or less than 100 mm (e.g., less than 30 mm) from reservoir 108. Alternatively, flow sensor 114 is positioned less than 5 mm from reservoir 108, and is positioned outside reservoir 108 such that nanoscale resistive element 200 is at least partially disposed within flow channel 120. As further described hereinbelow, for some applications, flow sensor 114 cleans itself and burn off any contaminants in the case of fouling; nonetheless, it is generally preferable to mitigate the chances of the fouling happening by isolating flow sensor 114 from the vaporized material as much as possible. Furthermore, it is advantageous as well to distance (e.g., isolate) flow sensor 114 from the rest of the electronic circuitry. Thus, if an unintentional fouling event occurs, flow sensor 114 can clean itself while the electronic circuitry will remain unharmed. Furthermore, in the case of an extreme fouling event (e.g., the entire nanoscale resistive element 200 becomes coated in a fouling mass), it is useful to have the sensor mounted directly in a flow channel of the vaporizer with a user-accessible port so that the user can clear some or all of the fouling mass with a rapid exhale (blow) into the device.

For example, in the configuration shown in FIG. 1, flow sensor 114 is positioned at least partially within flow channel 120, such that nanoscale resistive element 200 is exposed to airflow within flow channel 120. In this configuration if nanoscale resistive element 200 becomes severely fouled, a blow into the vaporizer from the user can clear some or all of the fouling mass. Flow sensor 114 as shown in FIG. 1 is also isolated from reservoir 108 by the positioning of heater 106 between reservoir 108 and flow sensor 114, and by being placed a sufficient distance (e.g., 5-100 mm). Additionally, sensing circuitry 115 is positioned within vaporizer housing 102 so as to be separated from the nanoscale resistive element positioned in flow channel 120.

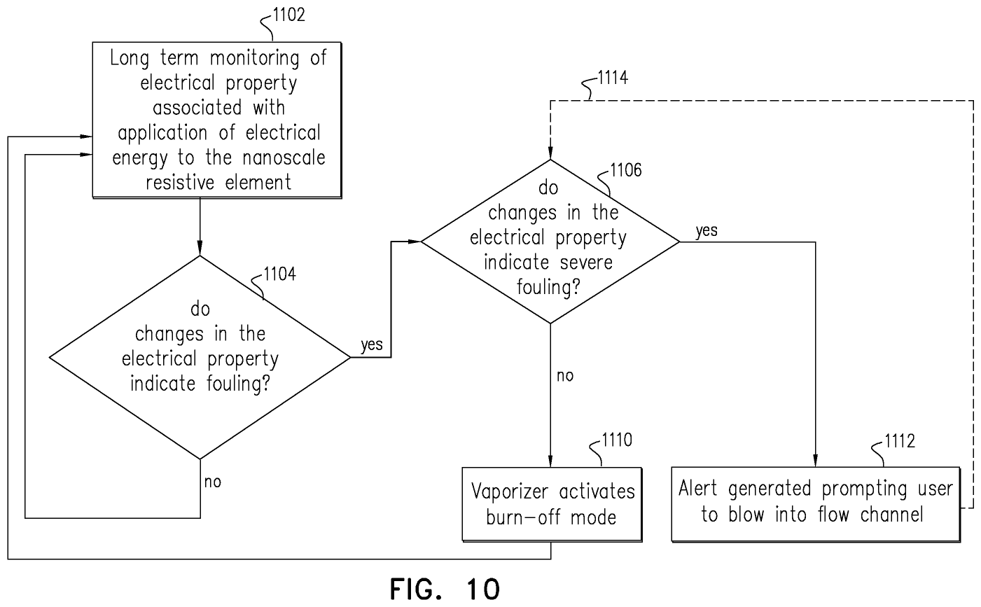

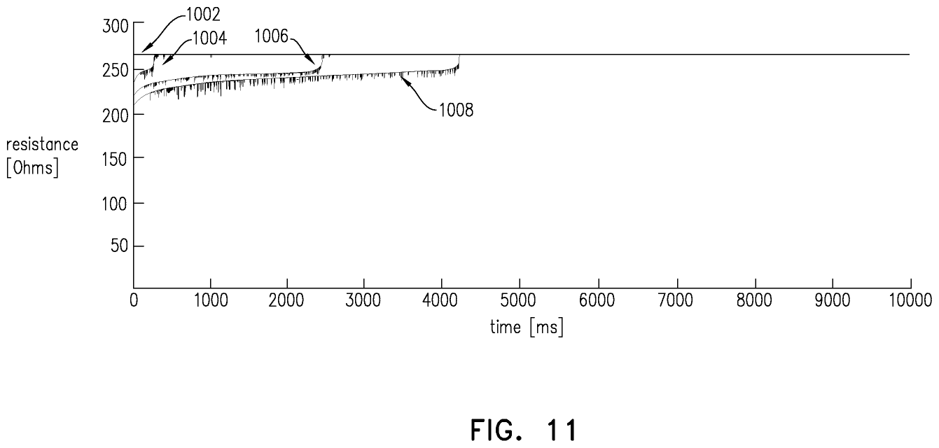

For some applications, flow sensor 114 may be configured with the option to have cleaning cycles, (also referred to herein as a “burn-off mode”), where the temperature of nanoscale resistive element 200 is increased to above the temperature of nominal operation in order to burn off any accumulated material, e.g., fouling mass, further described hereinbelow with reference to FIGS. 10-11. For some applications, sensing circuitry 115 is configured to increase the temperature of nanoscale resistive element 200 to at least 300 degrees Celsius and/or less than 1000 degrees Celsius during the cleaning cycle. In some applications, flow sensor 114 may perform a periodic cleaning cycle at fixed time intervals in order to maintain proper operation. In some applications, a cleaning cycle may occur in response to a change in state of the electronic cigarette. For example, when the electronic cigarette is connected to external power (e.g., to recharge the batteries) a cleaning cycle is enabled, and/or automatically activated, while utilizing the ability to draw more power without lowering the battery charge.

The characteristics of the heat loss in nanoscale resistive element 200 due to airflow depend on the ambient temperature that nanoscale resistive element 200 is exposed to. Therefore, in order to more accurately quantify the velocity and/or amount of airflow over nanoscale resistive element 200, the ambient temperature is measured so that any variation in the ambient temperature can be compensated for. In some applications, the ambient temperature is measured using a second nanoscale resistive element 200 configured for measuring temperature instead of velocity and/or amount of airflow.

Measuring temperature using nanoscale resistive element 200 is typically performed by biasing the nanoscale resistive element 200 to a level where Joule heating does not cause a change in temperature, and therefore causes negligible change in the resistance of nanoscale resistive element 200. In this state, the resistance of nanoscale resistive element 200 can be monitored in order to quantify the change in ambient temperature. More specifically, the TCR of nanoscale resistive element 200 provides a consistent and simple method of calculating temperature based on the measured resistance of nanoscale resistive element 200. Due to its smaller thermal mass and the optimized geometry of the support structure surrounding it, nanoscale resistive element 200 can provide fast temperature measurements, providing much higher frequency responses and much smaller settling times.

For some applications, the temperature measurement obtained using nanoscale resistive element 200 is used in conjunction with the velocity of the airflow measurement to reduce false positives for activation of the material vaporization. An example method for temperature-corrected puff detection follows. First, a temperature measurement is made using nanoscale resistive element 200. Second, a velocity of the airflow measurement is made using the same or a different nanoscale resistive element 200. (Alternatively, these first and second steps are performed in the reverse order.) Third, a value is determined, e.g., calculated or read (e.g., from a lookup table), using the temperature measurement to correct the velocity measurement, resulting in a temperature-compensated velocity value of the airflow. Fourth, the temperature-compensated velocity value is compared to a predetermined threshold to determine whether heater activation should occur or not. The above sequence is exemplary and other applications may include the same steps in different order, with a typical goal being to correctly identify user puffs in an environment of changing ambient temperature. Additionally, temperature correction calculations may be performed by a processor inside thermal flow sensor 114, or by a processor external to thermal flow sensor 114, such as a microcontroller 404 on controller PCB 110.

For some applications, the temperature measurement used for compensating for changes in ambient temperature is performed using the same nanoscale resistive element 200 that is used for measuring the velocity of the airflow. In this case, the nanoscale resistive element 200 is switched between (a) being operated in a high electrical current state and (b) being operated in a low electrical current state. As such, the velocity and/or amount of airflow is measured in the high current state, and ambient temperature is measured in the low current state. Due to the small size of the sensor, the settling times when switching between these two modes is significantly smaller than it is with larger sensor devices. Typically, using nanoscale resistive element 200, the temperature and velocity and/or amount of airflow can be accurately measured within 1 ms.

Using a single nanoscale resistive element 200, or two or more nanoscale resistive elements 200 within a single flow sensor 114, to measure velocity of the airflow and ambient temperature may provide cost and space savings. For example, a pressure-based solution often requires two separate sensors, as described hereinabove, each pressure-based sensor likely containing temperature measurement capability as well for temperature correction. The additional space and monetary cost for these requirements can be a significant difference for a high-volume consumer device like an electronic cigarette.

For some applications, puff detection may be performed with the single nanoscale resistive element 200 operated in the low current temperature measurement mode, and then the change in temperature due to the user sucking in cooler or warmer ambient air may be used to trigger the higher current velocity and/or amount of airflow measuring mode, thus saving power when in the puff detection state. For some applications, an alternative and/or additional activation mode is a user providing a short blow on the electronic cigarette, with either or both of the increased temperature and velocity of airflow used to trigger activation.

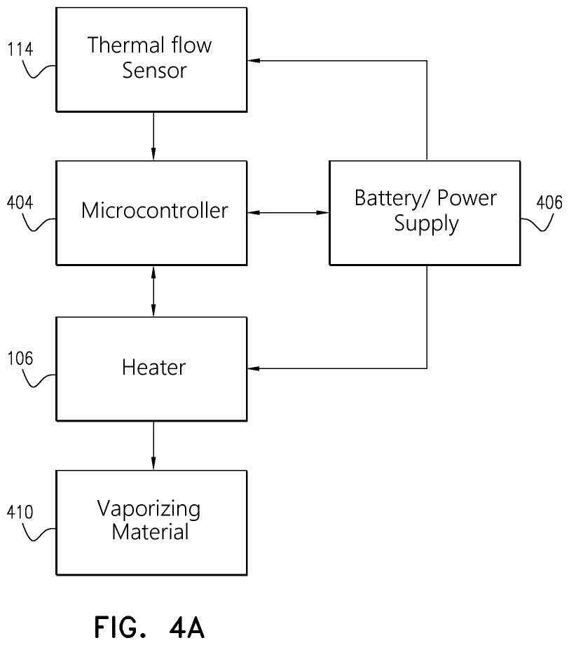

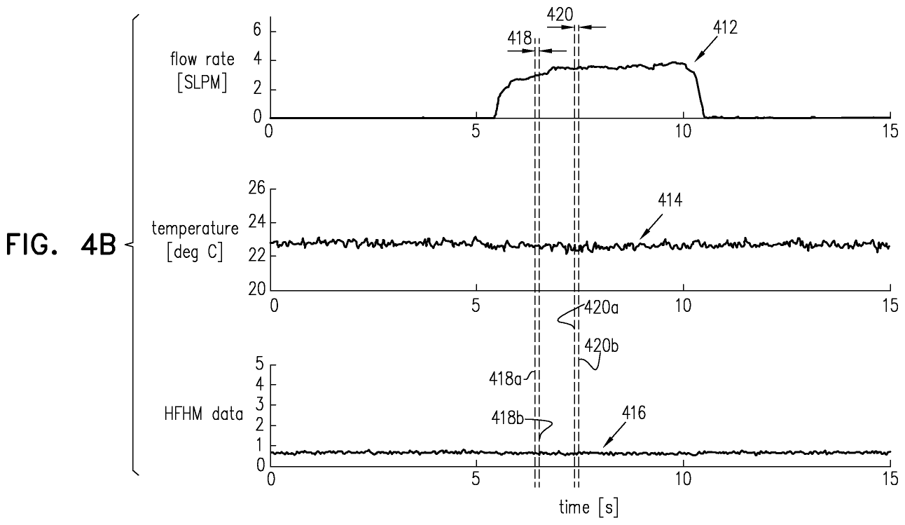

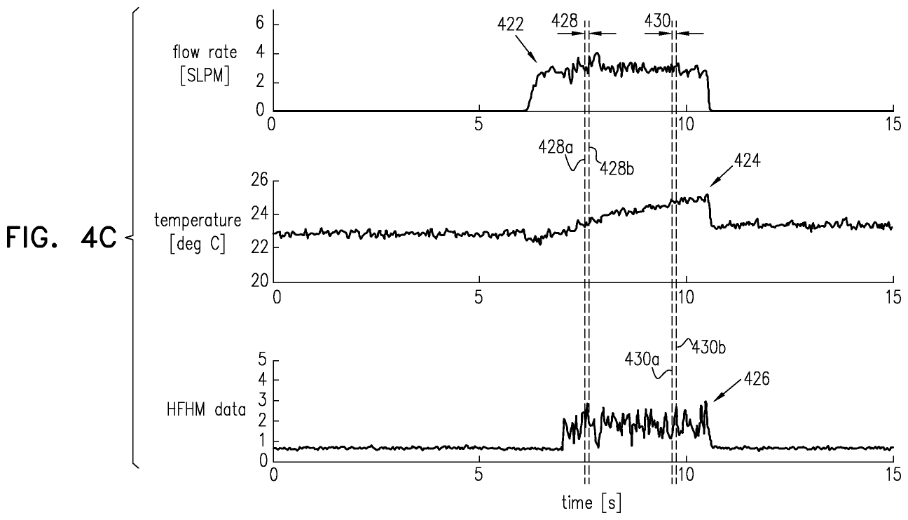

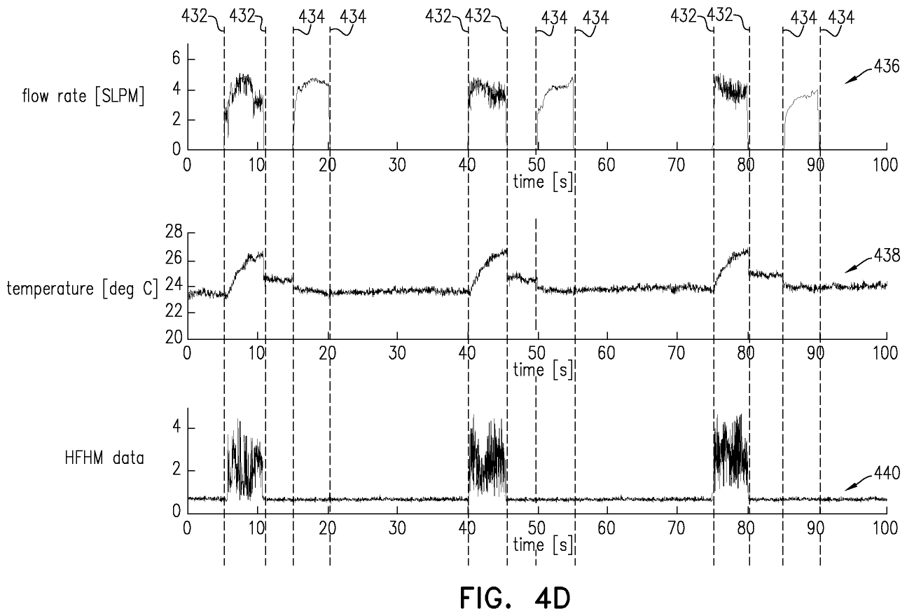

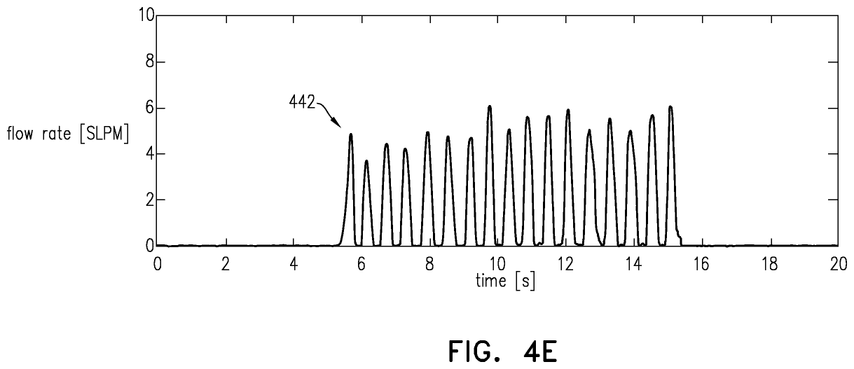

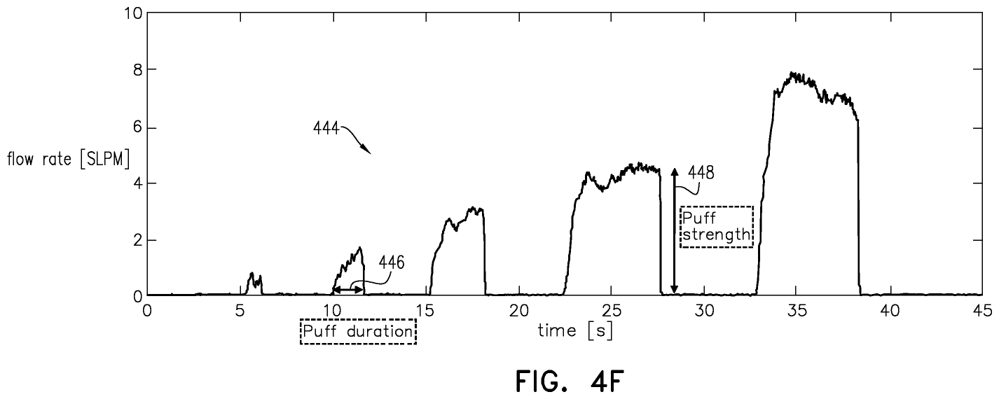

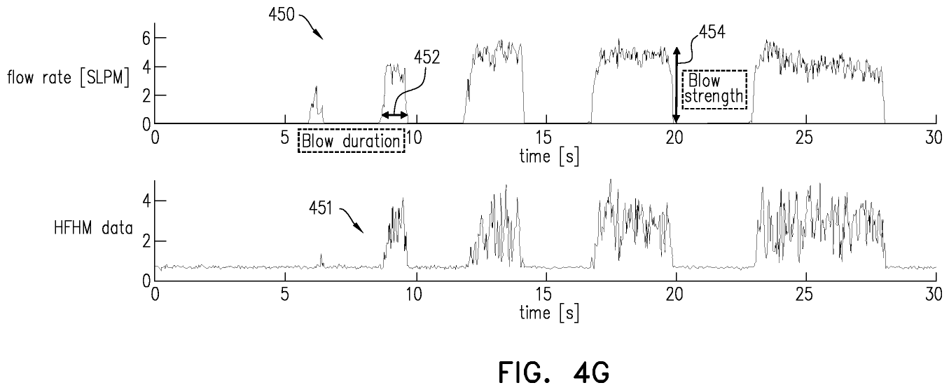

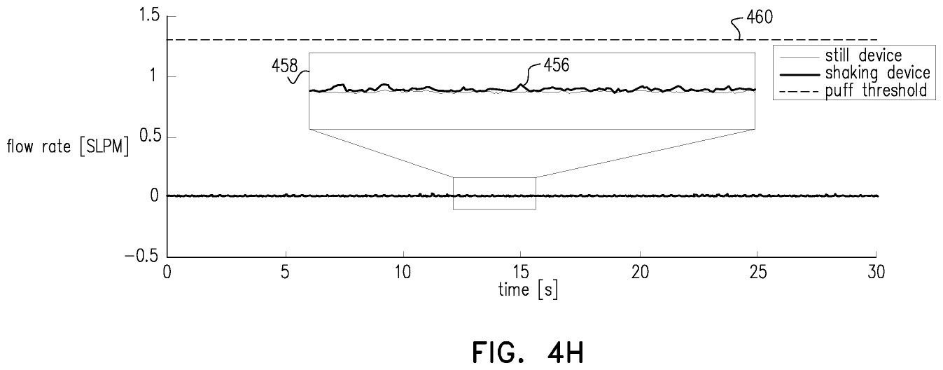

Reference is now made to FIG. 4, which is a block diagram depicting electronics relating to airflow detection and activation for an exemplary electronic cigarette, in accordance with some applications of the present invention. Thermal flow sensor 114 includes one or more nanoscale resistive elements 200, as described hereinabove, and provides sensor signals to a processor, e.g., microcontroller 404 that regulates activation of heater 106, which may be activated to heat a vaporizing material 410. Battery/power supply 406 provides power to thermal flow sensor 114, microcontroller 404, and heater 106. In some applications, thermal flow sensor 114 has an analog output, and in other applications, a digital output. The output type may depend on the characteristics of the microcontroller 404 and design of the system. For example, in some applications, microcontroller 404 has an I2C bus for connecting to various other peripheral devices in the system (e.g., an accelerometer), and thermal flow sensor 114 has a digital I2C connection to it. In some applications, microcontroller 404 contains an Analog to Digital Converter (ADC) that accepts analog signals, and thermal flow sensor 402 outputs analog signals.

Microcontroller 404 typically has software routines (typically referred to as firmware) running on it that work in conjunction with thermal flow sensor 114 to determine the state of the system and what functionality should be activated next. In the simplest use case, a user's puff on mouthpiece 112 of electronic cigarette 100 causes a measurement from the thermal flow sensor 114, e.g., the measured velocity of airflow within flow channel 120, to exceed a predetermined threshold value in the firmware, which, in turn, triggers activation of heater 106 and heating vaporizing material 410.

Activating heater 106 relatively quickly may provide an enhanced user experience. Thus, the measurement time of flow sensor 114 being relatively small decreases the time a user has to wait between his puff on mouthpiece 112 and heater 106 being activated. Other types of thermal flow sensors have longer measurement times due to larger thermal mass, requiring more time for temperature to stabilize and therefore do not provide the user with this enhanced user experience.

Additionally, an electronic cigarette typically has a very limited power budget due to being battery powered. Typically, an electronic cigarette is maintained in an unused, partially idle state where power consumption is minimal so that enough battery power is available for operation of the heater for brief periods of high-power vaporizing usage. Utilizing a low power sensor, such as MEMS-based thermal flow sensor 114, may help minimize overall power usage. Additionally, operating flow sensor 114 only periodically, as opposed to continuously, lowers the average power consumption.

MEMS-based thermal flow sensor 114 is especially suited for these low-power consumption purposes due to the small, i.e., nanoscale, size of the resistive element. This small size means less power is required to heat nanoscale resistive element 200 up to the required temperature, as there is less thermal mass present. Additionally, switching this type of sensor on and off can be done in a shorter, more efficient manner because the corresponding velocity of airflow signals and temperature signals settle faster than for sensors with larger thermal mass. For example, the MEMS-based thermal flow sensor 114 described hereinabove, which takes 1 ms of powered-up time to settle and measure, consumes five times less power than a comparable sensor that takes 5 ms of powered-up time to settle and measure. If both sensors from the above example have a continuous power usage rate of 5 mW, and are sampled every 100 ms, the average power usage will be 50 microwatts for the 1 ms sensor, compared to 250 microwatts for the 5 ms sensor.

Nanoscale resistive elements 200 used in the described applications have resistances typically between one and two orders of magnitude greater than a resistance of heater 106 used to vaporize vaporizing material 410. For example, nanoscale resistive elements 200 will typically have resistances of at least 50 Ohms and/or less than 200 Ohms at 20 degrees Celsius, while vaporizing heater 106 will typically have a resistance between 0.5 and 2 Ohms at 20 degrees Celsius. Accordingly, typical active power consumption for the nanoscale resistive element 200 will be at least 1 mW and/or less than 10 mW, while vaporizing heater 106 will consume 1-10 W when in an active state. As a result, it is not generally practical for a vaporizing heater to be used as an airflow sensor for a battery-operated device such as an electronic cigarette.

Furthermore, in some applications, MEMS-based thermal flow sensor 114 may provide an improvement in power consumption by replacing multiple devices within the electronic cigarette, each device having relatively high power consumption. For example, as previously described, some electronic cigarettes may use two absolute pressure sensors to detect a user's puff. Each of these pressure sensors may have a power consumption near or above the level of nanoscale resistive element 200, so replacing the two pressure sensors with one MEMS-based thermal flow sensor 114 typically results in reducing the power consumption to at least half the overall power consumption of the pressure-based solution.

For some applications, the temperature measurement of nanoscale resistive element 200 is used to actively modify the temperature of nanoscale resistive element 200 when nanoscale resistive element 200 is used for measuring velocity and/or amount of the airflow. For example, a modified CTA circuit is provided that, instead of maintaining the resistive element at a substantially constant absolute temperature, maintains the resistive element at a substantially constant differential temperature relative to the ambient temperature. By operating in this mode, the resistive element does not need to be at as high a temperature as the absolute temperature, and thus can be driven by less power, furthering the low power benefits of MEMS-based thermal flow sensor 114. For some applications, this mode of operation is achieved with a single resistive element that is switched between temperature measurement mode and velocity measurement mode, or two dedicated resistive elements (one for temperature measurement mode and one for velocity measurement mode). Additionally, the modified CTA operation described above may be used for a specific period of time (e.g., during puff detection), while the standard CTA operation is used for other periods of time (e.g., for more accurately measuring amounts of airflow once puff detection has already been triggered). Furthermore, the methods for modifying the CTA circuit operation may utilize analog and/or digital circuitry to perform the required operations.

Another benefit of the faster response of MEMS-based thermal flow sensor 114, in accordance with some applications, is the ability to accurately measure flow rate (i.e., the ability to measure flow rate enables another use beyond puff detection alone). With an accurate flow rate, the amount of consumed vaporizing material 410 can be determined and kept track of for dosage control or keeping track of how much material has been used. Both of these parameters can provide an enhanced user-experience, as dosage control can be used to ensure the user receives substantially the same dosage independent of the length of their inhalation, and tracking material usage can be useful for automated material ordering to ensure the user always has vaporizing material available. An exemplary usage of the ability to control dosage uses machine learning to calibrate based on the average puff duration of a user and to increase or decrease vapor amount for different lengths of puff to make sure the user gets a satisfying dose.

For some applications, since the flow rate measurements from nanoscale resistive element 200 can be used as an indication of the delivered quantity of vaporized material, MEMS-based thermal flow sensor 114 can be used as input to a real-time control program run by onboard microcontroller 404 that assists users looking to decrease their dependence on an addictive drug (e.g., nicotine) delivered in the vapor. Based on a custom program designed by or for the user, onboard microcontroller 404 can regulate the amount of nicotine delivered to the user per puff and/or per day in order to eventually decrease his dependence or ween him off nicotine entirely.

For some applications, this program may be implemented with a single reservoir of nicotine-based liquid to be vaporized. The user controls the settings for his device's per-puff and/or daily nicotine limit. Then, based on the data from MEMS-based thermal flow sensor 114, microcontroller 404 cut off power to the heater once the per-puff and/or daily limit quantity of nicotine is administered.

Alternatively, this program is implemented with two reservoirs of liquid—one containing nicotine and one that is nicotine-free. In this configuration, MEMS-based thermal flow sensor 114 can be used to switch the inhaled vapor from the nicotine to the nicotine-free liquid once the per-puff and/or daily limit of nicotine is met. This switching can be done by activating two separate heaters that exclusively vaporize one or the other of the two liquids, respectively, or any number of switching methods that can be controlled with valves or other mechanisms. This program has the added benefit of providing the user who wishes to quit with an uninterrupted puff experience, where nicotine vapor changes to nicotine-free vapor (e.g., gradually) due to real-time measurements from MEMS-based thermal flow sensor 114.

For some applications, the electronic circuitry inside vaporizer 20 that is responsible for operating MEMS-based thermal flow sensor 114 may also contain components that allow for wireless communication. This communication may utilize a variety of types of networks such as WiFi, Bluetooth, or Bluetooth Low Energy (BLE), and may transmit data to an external device, e.g., a mobile phone, tablet, or computer, and may work in conjunction with an application on the external device. Due to MEMS-based thermal flow sensor 114 being able to accurately detect puffs versus blows, further described hereinbelow, the velocity of airflow through flow channel 120, the amount of airflow, and the amount of material vaporized, the transmitted data may include information about the vaporizer usage such as: number of puffs, approximate amount of vaporizing material consumed per puff, and remaining vaporizing material in the reservoir. For some applications, this data may be relayed directly to (i) the user for information regarding their own usage of the device (e.g., in order to help them fight an addiction, as described hereinabove), (ii) a medical professional for information regarding a user's usage of the device, (iii) the manufacturer of the vaporizer, e.g., electronic cigarette, for data about how their devices are typically used by consumers, or (iv) to public health officials in order to monitor how large populations are using vaporizers, e.g., electronic cigarettes.

Taking into consideration what has been previously described, in some applications, there are two operating modes utilizing MEMS-based thermal flow sensor 114. In the first mode, nanoscale resistive element 200 is heated intermittently (e.g., one measurement every 100 ms) to detect puffs, enabled by both its fast settling time and its high frequency response. In the second mode, nanoscale resistive element 200 is constantly in low power puff detection mode until a puff is detected. In both cases, after a puff is detected, the flow sensor 114 may transition into a higher power puff/blow characterization mode to precisely measure the strength and duration of a puff or a blow, further described hereinbelow with reference to FIGS. 4F-G. Additionally, for some applications, this higher power mode is also activated when the user interacts with the electronic cigarette (e.g., using a combination of data from any or all of the MEMS-based flow sensors, a capacitive touch sensor, a temperature sensor, an accelerometer, etc.).

Furthermore, due to the real-time response of MEMS-based thermal flow sensor 114, the rate of change in flow and temperature (in conjunction with the absolute quantities) can be used to predict the onset or termination of a puff. This capability can be extended beyond the first order time derivative of temperature and velocity to higher order time derivatives as well. For some applications, microcontroller 404 may run algorithms based on this data in order to more efficiently actuate heater 106. For example, trends such as: a decreasing first derivative of velocity and an increasing first derivative of temperature (due to higher heater temperature at the end of a puff) can indicate that a puff is almost complete. These algorithms can be coupled with the aforementioned machine learning algorithms to more accurately detect the onset and termination of a specific users' puff in comparison to traditional techniques.

Reference is now made to FIGS. 4B-H, which are graphs showing various measurement signals from thermal flow sensor 114, in accordance with some applications of the present invention. Sensing circuitry 115 is configured to generate a sensor signal indicative of the measured velocity of the airflow within flow channel 120. For some applications, the processor, e.g., microcontroller 404, is configured to analyze a level of fluctuations in the sensor signal, and to determine a direction of the airflow within flow channel 120 in response to the level of fluctuations in the sensor signal. The fluctuations in the sensor signal may, for example, be identified by performing statistical moment analysis, e.g., standard deviation analysis, of the measured velocity of the airflow within flow channel 120. This is further described hereinbelow with reference to data curve 416 in FIG. 4B, data curve 426 in FIG. 4C, and data curve 440 in FIG. 4D. Due to the high frequency response of nanoscale resistive element 200, there is enough data in the sensor signal in order to perform the statistical moment analysis in real time by taking small time-windows, e.g., at least 1 microsecond-long time windows and/or less than 100 millisecond-long time-windows, and performing the statistical moment analysis, e.g., standard deviation analysis, on the data within each small time-window. To perform the analysis in real time, very small time-windows are used, each time window having enough data to analyze due to the high frequency response of nanoscale resistive element 200.

For some applications, the fluctuations in the sensor signal are generated due to a physical structure positioned to affect airflow at nanoscale resistive element 200 e.g., a physical structure that is separate from flow sensor 114 and placed in flow channel 120, or a physical part of flow sensor 114 other than nanoscale resistive element 200, e.g., substrate 214. For example, as shown in FIG. 2A, the part of substrate 214 that surrounds nanowire 208 is thin in order to minimize aerodynamic interference of the flow. Correspondingly, the part of substrate 214 that surrounds solder pads 212 (at the opposite end of flow sensor 114 from nanowire 208) is thicker for stability. In this manner, there is no disruption when airflow comes from the direction of nanowire 208, while airflow that comes from the direction of solder pads 212 is disrupted by the thicker part of substrate 214 before it reaches nanowire 208. The effect of the physical structure on the airflow at nanoscale resistive element 200 is reflected in the measured velocity of the airflow within flow channel 120. The inventors have realized that based on the relative positioning of the physical structure with respect to nanoscale resistive element 200, there are different effects on the sensor signal depending on which direction the air is flowing over nanoscale resistive element 200.

Typically, (a) when the direction of the airflow is such that air flowing through flow channel 120 reaches nanoscale resistive element 200 before the physical structure, the velocity of airflow sensor signal displays low levels of fluctuation, and (b) when the direction of the airflow is such that air flowing through flow channel 120 reaches the physical structure before nanoscale resistive element 200, the velocity of air flow sensor signal displays high levels of fluctuations due to eddies in the airflow caused by the physical structure.

Thus, as further described hereinbelow, the processor, e.g., microcontroller 404, may determine a direction of the airflow within the flow channel in response to the sensor signal, e.g., in response to a level of fluctuation in the sensor signal, e.g., as captured by statistical moment analysis of the sensor signal, by identifying (i) a high level of fluctuation in the sensor signal (as seen in FIG. 4C) as being indicative of airflow in a first direction with respect to the physical structure and (ii) a low level of fluctuation in the sensor signal (as seen in FIG. 4B) as being indicative of airflow in a second direction with respect to the physical structure, opposite the first direction. For some applications, the physical structure is positioned along flow channel 120 between nanoscale resistive element 200 and mouthpiece 112 of vaporizer 20, such that the processor identifies (i) airflow within flow channel 120 in the first direction (high level of fluctuation) as a blow, and (ii) airflow within flow channel 120 in the second direction as a puff. For some applications, the physical structure is positioned along flow channel 120 between nanoscale resistive element 200 and a distal end 126 of flow channel 120, such that the processor identifies (i) airflow within flow channel 120 in the first direction as a puff, and (ii) airflow within flow channel 120 in the second direction as a blow.

Typically, vaporizer 20, e.g., electronic cigarette 100, is configured to activate heater 106 in response to the measured velocity of airflow within flow channel 120 reaching a threshold value. The processor, e.g., microcontroller 404, being able to accurately identify a direction of airflow with flow channel 120, as described hereinabove, helps to prevent improper heater activation, e.g., improper activation of heater 106 in response to the velocity of airflow within flow channel 120 reaching the threshold value due to a user blowing into mouthpiece 112. Thus, based on the determination of airflow direction, vaporizer 20, e.g., electronic cigarette 100 (i) activates heater 106 when the measured velocity of airflow within flow channel 120 during a puff reaches the threshold value, and (ii) does not activate the heater when the processor identifies the airflow within flow channel 120 as a blow. As described hereinabove, with an accurate flow rate, the amount of consumed vaporizing material 410 can be determined and tracked for dosage control or for keeping track of how much material has been used. Avoiding improper activations of heater 106 by accurately determining the direction of flow within flow channel 120 additionally helps to make sure that the determined amount of consumed vaporizing material is accurate.

Reference is now made specifically to FIG. 4B, which shows three data curves 412, 414, and 416 captured during a puff, which correspond respectively to the flow rate, the temperature signal, and a parameter referred to herein as High-Frequency-Higher-Moment (HFHM) data, which is calculated based on the statistical moment analysis of the flow rate, in accordance with some applications of the present invention. (The HFHM data generally correspond to analysis of statistical moments that are higher than the first-order moment (i.e., the mean); for example, the HFHM data may correspond to the real-time standard deviation of the flow rate.) It is noted that in this particular example, the physical structure is positioned along flow channel 120 between nanoscale resistive element 200 and mouthpiece 112, such that airflow due to a puff reaches nanoscale resistive element 200 before the physical structure. Due to a puff causing ambient air to flow past nanoscale resistive element 200, the temperature within flow channel 120 stays generally constant for the duration of a puff, as shown by data curve 414. Data curve 416 shows the HFHM data analysis of flow rate curve 412. Time-window 418 (shown between dashed lines 418a and 418b) and time-window 420 (shown between dashed lines 420a and 420b) represent two examples of the small time-windows in which the HFHM data is calculated. For each time-window, the HFHM data is calculated for the values of the flow rate within that time-window. It is noted that for illustrative purposes time-windows 418 and 420 are depicted significantly larger than they are actually are. As is shown by data curve 416, the HFHM data corresponding to the increased airflow flowing over nanoscale resistive element 200 and subsequently the physical structure, is relatively constant. For some applications, the HFHM data is the standard deviation, in which case the HFHM data for each time window represents how much the flow rate values fluctuate around a local mean value for that time window. As shown, the HFHM data stays relatively low and relatively constant throughout the measurement.

Reference is now made specifically to FIG. 4C, which shows three data curves 422, 424, and 426 captured during a blow, which correspond respectively to the flow rate, the temperature signal, and the High-Frequency-Higher-Moment (HFHM) data, calculated based on the statistical moment analysis of the flow rate, in accordance with some applications of the present invention. It is noted again that in this particular example, the physical structure is positioned along flow channel 120 between nanoscale resistive element 200 and mouthpiece 112 such that airflow due to a blow reaches the physical structure before nanoscale resistive element 200. Due to a blow causing air from a user's body to flow past nanoscale resistive element 200, the temperature within flow channel 120 rises throughout the duration of a blow, as shown by data curve 424. Data curve 426 shows the HFHM data analysis of flow rate curve 422. Time-window 428 (shown between dashed lines 428a and 428b) and time-window 430 (shown between dashed lines 430a and 430b) represent two examples of the small time-windows in which the HFHM data is calculated. For each time-window the HFHM data is calculated for the values of the flow rate within that time-window. It is noted that for illustrative purposes, time-windows 428 and 430 are depicted significantly larger than they are actually are. As is shown by data curve 426, the HFHM data corresponding to the increased airflow flowing over the physical structure and subsequently nanoscale resistive element 200, varies substantially. For some applications (and as in the data shown), the HFHM data is the standard deviation, in which case the HFHM data for each time window represents how much the flow rate values fluctuate around a local mean value for that time window. As shown, although the HFHM data itself fluctuates, it is generally higher at most points than the corresponding HFHM data for airflow in the other direction (represented by data curve 416 in FIG. 4B), indicating real-time fluctuations in the flow rate due to the eddies caused by airflow over the physical structure.

Reference is now made specifically to FIG. 4D, which shows data curves 436, 438, and 440, captured during a series of blows and puffs, which correspond respectively to the flow rate, the temperature signal, and the High-Frequency-Higher-Moment (HFHM) data, calculated based on the statistical moment analysis of the flow rate, in accordance with some applications of the present invention. FIG. 4D highlights the ability to accurately distinguish between puffs and blows based on analysis of the sensor signals by showing the contrasting signal characteristics of airflow in the two different directions. The segments of the data curves that appear between the sets of dashed lines 432 correspond to blows, and the segments of the data curves that appear between the sets of dashed line 434 correspond to puffs. It is noted again that this is a particular example where the physical structure is positioned between nanoscale resistive element 200 and mouthpiece 112. The high level of signal fluctuations that are characteristic of the airflow flowing over the physical structure before nanoscale resistive element 200 are apparent in each data segment that corresponds to a blow in both flow rate data curve 436 and HFHM data curve 440. The low level of signal fluctuations that are characteristic of the airflow flowing over nanoscale resistive element 200 before the physical structure are apparent in each data segment that corresponds to a puff in both flow rate data curve 436 and HFHM data curve 440. Temperature data curve 438 shows a rise in temperature during each blow, and a generally constant cooler temperature during each puff.

Reference is now made specifically to FIG. 4E, which shows a data curve 442 from an experiment carried out by the inventors in which 17 short puffs were performed within about 10 seconds, at a puff rate of less than 1 Hz. The clear distinct peaks in the sensor signal corresponding to each of the 17 puffs indicates that nanoscale resistive element 200, due to its high frequency response, is sensitive to even very short quick puffs and that flow sensor 114 can accurately measure very short puffs, even when they are consecutive.

Reference is now made specifically to FIG. 4F, which shows a data curve 444 corresponding to flow rate, obtained in an experiment carried out by the inventors in which a series of increasingly stronger and longer simulated puffs were performed. As described hereinabove, flow sensor 114 may transition into a higher power puff/blow characterization mode to precisely measure the strength and duration of a puff or a blow. As shown in data curve 444, (a) the width at the widest point of each puff signal, represented by double-headed arrow 446, indicates the duration of the puff, and (b) the height at the highest point of each puff signal, represented by double-headed arrow 448, indicates the strength of the puff. Being able to characterize a puff based on duration and strength may help with long-term monitoring of vaporizer 20. For example, if vaporizer 20 is generally used by a single user, and the puffs from that user are generally consistent in duration and strength, then a detected change in the duration or strength of a puff may be an indication that a different user is using the device, an indication of a medical issue, or an indication that there may be something wrong with the vaporizer, e.g., a blockage in the flow channel. For some applications, the vaporizer 20 records the first one or few puffs of a user. This data can be used to detect a potential blockage during usage. For some applications, this detection may be performed by recording flow over time and establishing a flow rate profile baseline for that user. Deviations from such baseline may indicate a potential blockage. Additionally or alternatively, being able to characterize a puff based on duration and strength may also serve to identify a true puff versus a false positive activation signal that is caused by a non-puff airflow, such as for example, a car door closing in a manner that causes some airflow in the flow channel, or a gust of wind.

Reference is now made specifically to FIG. 4G, which shows data curves 450 and 451 corresponding respectively to flow rate and corresponding HFHM data from an experiment carried out by the inventors in which a series of increasingly stronger and longer simulated blows were performed. As shown in data curve 450, (a) the width at the widest point of each blow signal, represented by double-headed arrow 452, indicates the duration of the blow, and (b) the height at the highest point of each puff signal, represented by double-headed arrow 454, indicates the strength of the blow.

Reference is now made specifically to FIG. 4H, which is a data graph showing flow rate measured in response to shaking electronic cigarette 100 during an experiment carried out by the inventors. As observed from data curve 456 (a segment of which is shown in enlarged-view 458), in response to the shaking, thermal flow sensor 114 measured small fluctuations in airflow that were well below the puff threshold value, represented by dashed line 460. Due to flow channel 120 being so small, a substantial pressure differential at the opening to flow channel 120, e.g., at mouthpiece 112, is needed in order to cause airflow through the channel that is strong enough to trigger activation of the heater, e.g., the pressure differential caused by a user inhaling through or blowing into flow channel 120. Thus, due to vaporizer 20 activating the heater, e.g., heater 106, in response to an airflow measurement, rather than in response to a pressure measurement, improper activations of the heater in response to ambient pressure changes, or even shaking of vaporizer 20, are avoided. For example, in a pressure-sensor-based vaporizer, it is possible that ambient pressure changes, such as for example an ambient pressure change in response to the closing of a car door, may cause an improper activation of the vaporizer heater in response to the change in pressure. Vaporizer 20 is not as susceptible to improper positive heater activation in response to ambient pressure changes since such pressure changes are not strong enough to cause substantial airflow through the narrow flow channel.

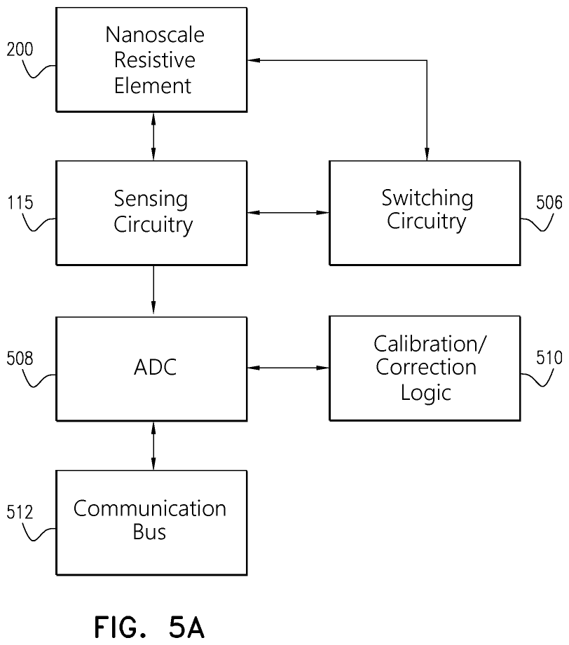

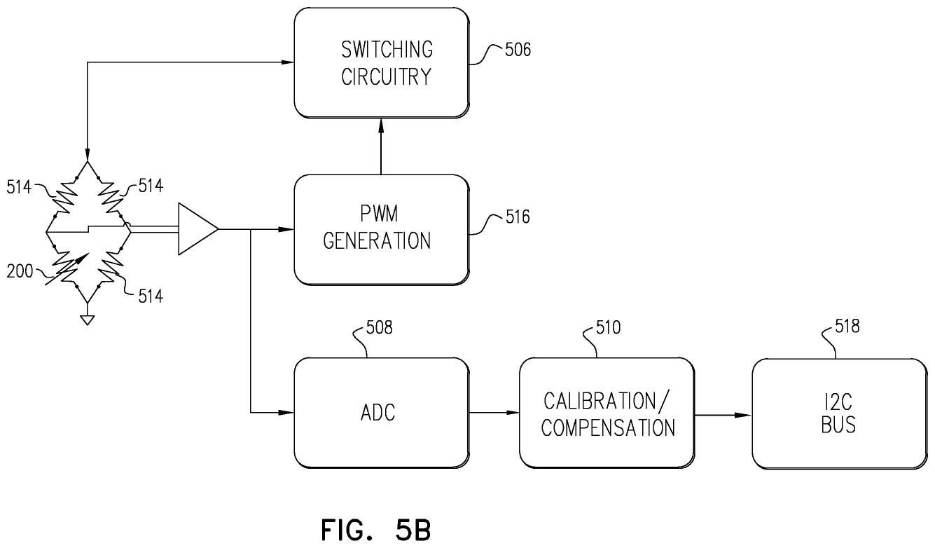

Reference is now made to FIG. 5A, which is a block diagram depicting the electronics of MEMS-based thermal flow sensor 114, in accordance with some applications of the present invention. Sensing circuitry 115 provides power to nanoscale resistive element 200, i.e., to either nanoscale wire(s) 208 or nanoscale film resistive element(s) 300, according to the modes of operation previously described (including CCA, CVA, and/or CTA), and generates sensing signals for indicating temperature and velocity and/or amount airflow. Additionally, sensing circuitry 115 can also be operated in pulsed or linear operation mode when providing power to nanoscale wire(s) 208 or nanoscale film resistive element(s) 300. The sensing signal(s) generated by sensing circuitry 115 indicate temperature and airflow levels using principles described hereinabove. Switching circuitry 506 provides capability to switch nanoscale wire(s) 208 or nanoscale film resistive element(s) 300 between different modes and/or to different biasing levels. For some applications, switching circuitry 506 operates with a single nanoscale wire 208 or nanoscale film resistive element 300 and switches between two modes and/or biasing levels to provide (i) temperature and (ii) velocity and/or amount of airflow measurement capabilities. For other applications, switching circuitry 506 operates with two or more nanoscale resistive elements 200, i.e., two or more nanoscale wires 208 or nanoscale film resistive elements 300, and switches between two modes and/or biasing levels to provide (i) temperature and (ii) velocity and/or amount of airflow measurement capabilities.

For some applications analog to digital converter (ADC 508) provides conversion of analog signals from sensing circuitry 115 to digital signals. Depending on the accuracy and sensitivity required in the system, an ADC 508 with resolution of 12-bit to 24-bit is typically used, and may be a flash, successive-approximation, delta-sigma, or other type. In some applications, ADC 508 is used in conjunction with calibration/correction logic 510 to provide a more accurate digitally-converted measurement. For example, in some applications, calibration data may include characteristics of nanoscale wire(s) 208 or nanoscale film resistive element(s) 300 measured during or after production that are used to provide more consistent results by compensating for small differences in the nanoscale resistive elements 200 that occur during the manufacturing process. In some applications, the calibration data also includes characterization of various portions of the circuitry that are measured during or after manufacturing/integration of the electronics with the nanoscale wire(s) 208 or nanoscale film resistive element(s) 300. These characteristics may include offsets, slopes, non-linearities, and other characteristics that can be determined and compensated for to provide more accurate temperature and airflow measurements.

In some applications, the correction provided by calibration/correction logic 510 uses the calibration data described hereinabove to correct for errors and provide more accurate measurements. In some applications, the correction provided by calibration/correction logic 510 also provides correction based on ambient conditions. For example, as described hereinabove, ambient temperature measurements can be used to correct and provide a more accurate velocity and/or amount of airflow measurement.

Communication bus 512 provides connection and communication to external devices that support operation of vaporizer 20, e.g., electronic cigarette 100, and typically utilizes a digital communication protocol such as an Inter-Integrated Circuit (I2C) protocol or a Serial Peripheral Interface (SPI) protocol. An exemplary method of operation utilizing communication bus 512 in vaporizer 20, e.g., electronic cigarette 100, is as follows. First, an external device (e.g., microcontroller 404 controlling the operation of electronic cigarette 100) issues a command over communication bus 512 requesting (i) a velocity and/or amount of airflow and/or (ii) a temperature measurement from the flow sensor 114. Second, flow sensor 114 receives this command over communication bus 512 and performs the measurement operations as described hereinabove. Third, after a period of time (e.g., 1 ms settling and conversion time), microcontroller 404 receives a signal over communication bus 512 indicating that a measurement is ready, and/or sends a request over communication bus 512 for the measurement data. Fourth, flow sensor 114 sends measurement data over communication bus 512 to microcontroller 404.

Reference is now made to FIG. 5B, which is a block diagram depicting a specific implementation of the electronics of MEMS-based thermal flow sensor 114 as shown in FIG. 5A, in accordance with some applications of the present invention. Nanoscale resistive element 200 is shown in FIG. 5B in a bridge configuration which includes resistors 514 to provide a differential signal that varies according to the resistance of nanoscale resistive element 200, and is used for driving nanoscale resistive element 200 to different power levels according to the airflow in a CTA configuration, e.g., the resistance of nanoscale resistive element 200 is forced to remain at a constant value (indicating that it is at a constant temperature), in order for the bridge to remain in a balanced state (differential voltage=0 V); the more airflow there is in the flow channel, the more current is driven into nanoscale resistive element 200 in order to maintain the constant temperature. Pulse-Width Modulation (PWM) generator 516 is an example of a specific implementation of sensing circuitry 115 of FIG. 5A. I2C bus 518 is a particular example of communication bus 512 of FIG. 5A.

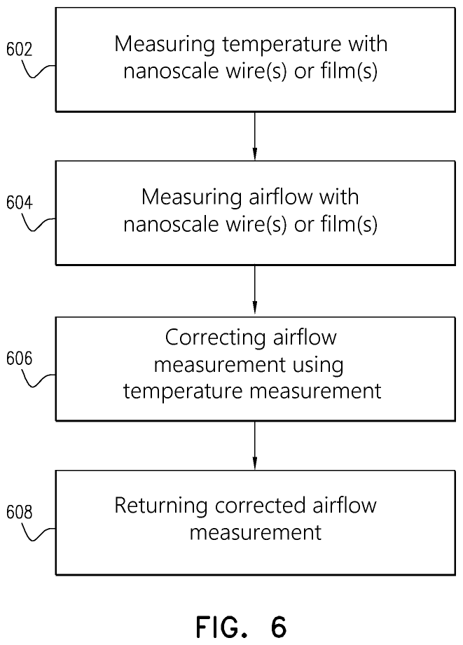

Reference is now made to FIG. 6, which is a flow chart for an example application of a method of using MEMS-based thermal flow sensor 114 for measuring velocity and/or amount of airflow and temperature, in accordance with some applications of the present invention. At step 602, temperature is measured with nanoscale wire(s) 208 or nanoscale film resistive element(s) 300. At step 604, airflow, i.e., velocity and/or amount of the airflow, is measured with nanoscale wire(s) 208 or nanoscale film resistive element(s) 300. At step 606, the airflow measurement is corrected using the temperature measurement from step 602. At step 608, the corrected airflow measurement is returned.

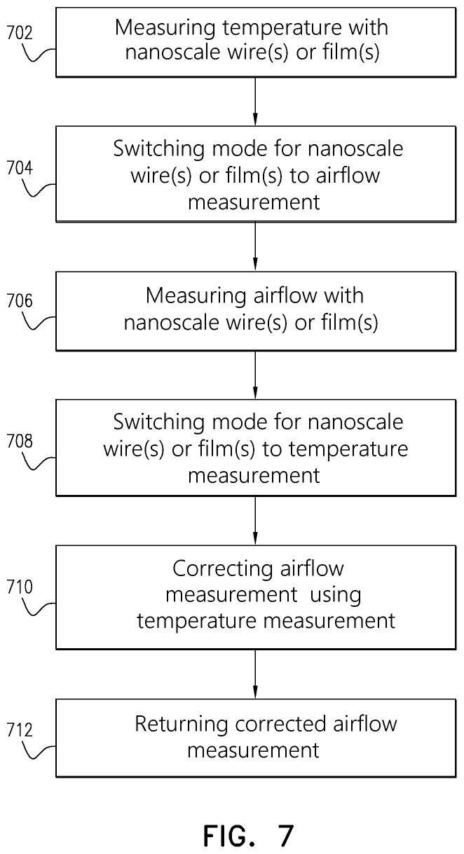

Reference is now made to FIG. 7, which is a flow chart for an example embodiment of a method of using MEMS-based thermal flow sensor 114 for measuring airflow and temperature using a single nanoscale resistive element 200, in accordance with some applications of the present invention. At step 702, temperature is measured with nanoscale wire(s) 208 or nanoscale film resistive element(s) 300. At step 704, the mode of operation for the nanoscale resistive element 200 is switched to be configured for velocity of airflow measurement. At step 706, velocity of airflow is measured with nanoscale wire(s) 208 or nanoscale film resistive element(s) 300. At step 708, the mode of operation for nanoscale resistive element 200 is switched to be configured for temperature measurement. At step 710, the velocity of the airflow measurement is corrected using the temperature measurement from step 702. At step 712, the corrected airflow measurement is returned.

Correct integration of MEMS-based thermal flow sensor 114 with the electronic cigarette's body is provided for proper operation of the system. In some applications, MEMS-based thermal flow sensor 114 including its electronics (i.e., sensing circuitry 115) and all supporting components are in a discrete package installed in the body of electronic cigarette 100, e.g., within flow channel 120 of electronic cigarette 100. In another application, MEMS-based thermal flow sensor 114 uses the body of electronic cigarette 100 as the package. In yet another application, nanoscale resistive element 200 and sensing circuitry 115 are separately installed so that the components that make up the entirety of MEMS-based thermal flow sensor 114 can be in different locations.

For typical operation, the nanoscale resistive element portion of the MEMS-based thermal flow sensor 114 is installed in flow channel 120 that leads directly from mouthpiece 112 to the surrounding environment external to electronic cigarette 100. In some applications, electronic cigarette 100 contains a detachable cartridge (configuration not shown). It is desirable to ensure that there is no or minimal leakage between the cartridge and the main body of electronic cigarette 100. In some applications, this can be done using a seal, such as a rubber seal or other ways known to those skilled in the art to ensure no or minimal leakage. The seal can extend the full width of the cartridge, or in other embodiments can extend less than the width of the cartridge. In some embodiments, the seal is an O-ring.

A part of providing a satisfactory user experience is ensuring that the suction pressure required to inhale the desired amount of vaporizing material 410 is neither too high nor too low. In some applications, a rubber seal that has a small opening may be used to regulate the suction pressure. In this case, the flow rate in the channel that leads to MEMS-based thermal flow sensor 114 will be slightly lower than the inhaling flow rate. Alternatively or additionally, regulating the suction pressure is achieved by varying the dimensions of flow channel 120 leading to the MEMS-based thermal flow sensor 114 or by splitting flow channel 120 into multiple channels.

In some applications, decreasing flow channel pressure serves as a way to ensure that the user gets the right suction pressure for an improved user experience.

Flow channel 120 connecting mouthpiece 112 to the surrounding environment can exit through openings on the sides of electronic cigarette 100. In other embodiments, in order to avoid blockage by the user, e.g., the user's hand while he is holding electronic cigarette 100, flow channel 120 can exit at the back of the device. When the user applies suction pressure to mouthpiece 112, all or some of the flow will flow to flow channel 120 and will be measured by MEMS-based thermal flow sensor 114.

In some embodiments, the complete MEMS-based thermal flow sensor 114 is located within flow channel 120. Alternatively, in other applications, a portion of the sensor 114 (e.g., only nanoscale resistive element 200) is located within flow channel 120. In some applications, the packaging of MEMS-based thermal flow sensor 114 includes a portion of the wall of flow channel 120 and when coupled to the body of electronic cigarette 100, flow channel 120 is completed.

In some applications, in order to protect nanoscale resistive element 200 from particles that might enter flow channel 120 due to the user's suction, a filter is installed in any portion of flow channel 120. In some applications, flow channel is shaped in a way that allows large particles to flow in a secondary flow path that is not in contact with the sensor. For example, in some applications, flow channel 120 has a radius of curvature such that the large and small particles are separated by their differences in inertia.

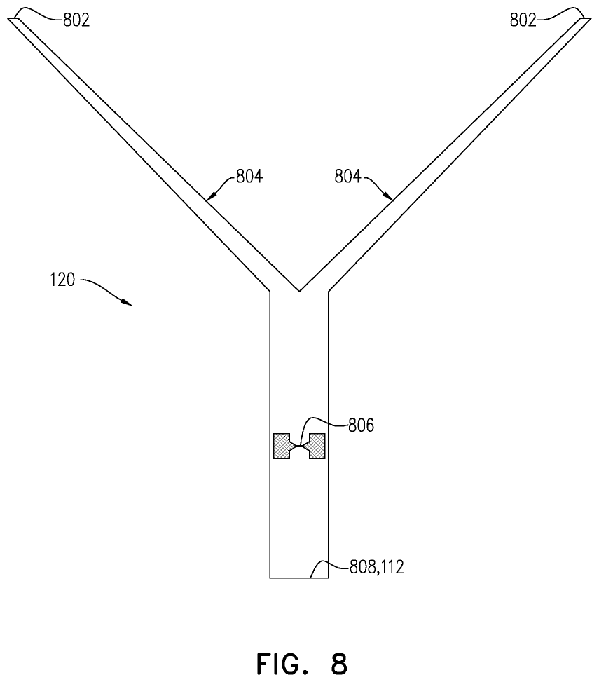

Reference is now made to FIG. 8, which is a schematic illustration of flow channel 120, in accordance with some applications of the present invention. In some applications, flow channel 120 is shaped in a diverging shape where an inlet 802 is the smallest portion of the diverging channel. At the opposite end of flow channel 120 is outlet 808, i.e., at mouthpiece 112 of electronic cigarette 100. Inlet 802 may inhibit the entrance of larger particles into flow channel 120 and ensure that particles that do enter flow channel 120 will not get stuck in the channel as the cross-section of flow channel 120 is continually increasing as the particles move the flow channel. This diverging shape will also decrease the particle velocity due to the increase in cross-sectional area, thus ensuring that particles that make contact with nanoscale resistive element 200 will have less momentum than they had upon entering flow channel 120. In some applications, flow channel 120 is diverging along the entire length of flow channel 120. Alternatively, in other applications, the diverging section is a portion of flow channel 120.

For some applications, such as is shown in FIG. 8, since the inlet 802 of flow channel might be small so as to inhibit particles above a certain size from entering flow channel 120, flow channel 120 may have multiple diverging inlet channels 804, each with its own inlet 802, leading to the main channel and outlet 808. In these designs, the flow sensor 806 may be placed downstream of diverging inlet channels 804, filtration, or other features meant to decrease the risk of large particulate matter impacting the sensor.

In some applications, if flow channel 120 or inlet channels 804 get blocked, an indication is provided to the user such as a blinking/colored light, sound, LCD display error, sound vibration, app notification, or other observable indicator. The user might be instructed to blow into the device to release trapped particles or any liquid or residues accumulated in the channel. In some applications, the user activates the device by a blowing action, which might also serve to purge flow channel 120.

As described hereinabove, for some applications, flow sensor 114 oscillates between a flow-measurement mode and a temperature-measurement mode. Additionally to the uses of the temperature as described hereinabove and below with reference to real-time temperature compensation of the flow measurement and determining direction of airflow within the flow channel, the temperature data from nanoscale resistive element 200 may be used to monitor the health of battery 406 during operation of vaporizer 20, as well as during charging of the battery, by detecting overheating of the battery. Likewise, performance monitoring via the temperature data can be applied to the sensing circuitry 115 inside vaporizer 20, as well as to heater 106, in order to detect any degradation or abnormal functioning of a component. For example, the temperature data from nanoscale resistive element 200 may be used as a closed-loop feedback for heater 106, e.g., if microcontroller 404 activates heater 106, the temperate data from nanoscale resistive element 200 may be used to determine if indeed heater 106 was activated and/or was properly heated to the desired temperature for vaporizing the material. For some applications, MEMS-based thermal flow sensor 114 records temperature, and the device uses this data to calculate the ideal heater temperature for best user experience. Nanoscale resistive element 200 may provide an indication of the temperature of heater 106 by indirect temperature measurement at the location of nanoscale resistive element 200, and therefore depends on the location of flow sensor 114 within vaporizer 20.

Alternatively or additionally to the analysis of the airflow signal as described hereinabove for determining a direction of the airflow, the following applications for determining a direction of the airflow within flow channel 120 are provided:
In some applications, two MEMS-based thermal flow sensors 114 are used in conjunction and placed within a predetermined distance (d) from each other along flow channel 120. A temperature or flow perturbation that is sensed by the first sensor 114 is detected by the second sensor 114 after an amount of time (Dt). The predetermined distance (d) between the two sensors 114 divided by the time (Dt) is used to determine the velocity (v=d/Dt). This also allows sensing the flow direction by knowing which sensor detects the perturbation first.
In some applications, a small obstruction is placed in front or after the MEMS-based thermal flow sensor 114. Airflow over the obstruction often causes vortex shedding that can be sensed by the sensor 114, providing another way to detect the flow direction.
In some applications, MEMS-based thermal flow sensor 114 is manufactured with multiple nanoscale resistive elements 200 that are aligned at certain offset angles from one another. Based on the respective signals from these differently-oriented nanoscale resistive elements 200, MEMS-based thermal flow sensor 114 can determine the direction of one-dimensional airflow through flow channel 120.
In some applications, the flow direction is deduced by the difference in temperature caused by suction (cooling) or blowing (heating), given that the ambient temperature is usually lower than body temperature. If the ambient temperature is higher than the body temperature, a cooling of the sensor indicates a flow coming from the mouthpiece (blowing).

In some applications, a one-way valve or check valve is included in flow channel 120 of electronic cigarette 100. The valve may be a flap valve, a ball check valve, a diaphragm check valve, or other applicable mechanism known to those skilled in the art. In some applications, the valve is a portion of another component in flow channel 120. For example, the one-way valve may be a rubber flap valve that is integrated with a rubber seal configured to seal the channel as previously described. By providing a one-way valve in flow channel 120, it can be ensured that any significant flow detected by nanoscale resistive element 200 is of a certain direction.

In some applications, a user communicates with electronic cigarette 100 by tapping on the inlet/outlet of flow channel 120. This causes a distinct pulse of airflow that can be sensed by MEMS-based thermal flow sensor 114, which exhibits high sensitivity at these very low velocities. For example, two taps within a certain time period can be interpreted by the controller as instructions to perform a certain operation, while three or four taps within a certain time period can be associated with other operations. Use of this capability can be used to actuate other functions of electronic cigarette 100 as well, such as to wake from a sleep mode, to indicate a status (e.g., battery charge level), to change a mode of operation, or other features that may be desirable for the user.

In some applications, the use of MEMS-based thermal flow sensor 114 to capture user inputs may replace or augment an accelerometer or other type of sensor within electronic cigarette 100 that may otherwise have been used to capture user inputs and/or instructions. For example, in some electronic cigarettes, an accelerometer is used to capture user inputs and act on it or provide information as previously described. The use of MEMS-based thermal flow sensor 114 to capture the same or similar inputs while also gathering other information such as puff detection, flow rate, and temperature, may provide substantial cost, power, and space savings for electronic cigarette 100. Additionally, MEMS-based thermal flow sensor 114 in these applications is able to differentiate between intentional and unintentional user inputs since it has a more focused activation method than an accelerometer.

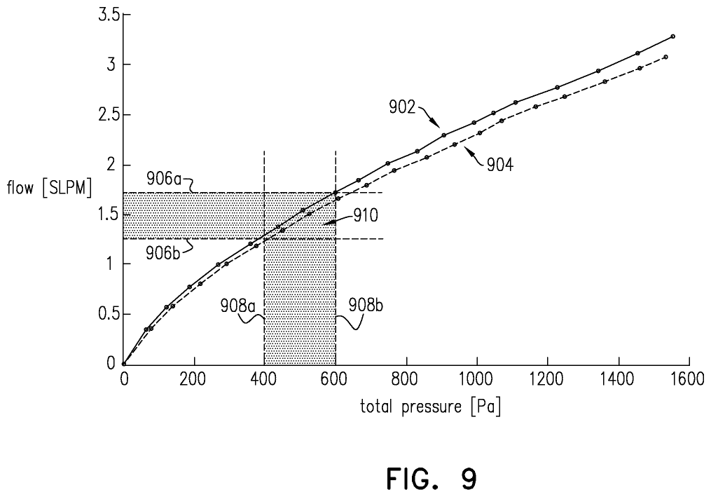

Reference is now made to FIG. 9, which is a data graph showing two data curves 902 and 904 depicting flowrate versus pressure within the flow channel in an experiment carried out by the inventors, in accordance with some applications of the present invention. Data curve 902 represents the airflow versus pressure curve for a conventional electronic cigarette containing a pressure-sensor. Data curve 904 represents the airflow versus pressure for the same physical electronic cigarette, in which the inventors removed the pressure-sensor and installed MEMS-based thermal flow sensor 114 instead. The flow rate values between dashed lines 906a and 906b represent a range of values for the airflow velocity-based puff detection threshold, e.g., at least 1.3 SLPM and/or less than 1.7 SLPM. The pressure values between dashed lines 908a and 908b represent a range of values for the pressure-based puff detection threshold of electronic cigarette (prior to having the pressure sensor swapped for flow sensor 114), e.g., at least 400 Pa and/or less than 600 Pa. As observed by the inventors, the airflow versus pressure curves with the two different sensors appear very close together in the overlapping region 910 of the two puff thresholds, leading to the conclusion that using MEMS-based thermal flow sensor 114 to sense airflow in the flow channel of a vaporizer, e.g., electronic cigarette, does not change the overall airflow versus pressure characteristics of the vaporizer, e.g., an electronic cigarette.

Furthermore, for some applications, the processer, e.g., microcontroller 404 may be configured to analyze the sensor signal indicative of the measured velocity of the airflow within flow channel 120, as generated by sensing circuitry 115, and determine a differential pressure within flow channel 120. This allows MEMS-based thermal flow sensor 114 to be used in a vaporizer that is designed to work with a conventional pressure sensor and to activate a heater for vaporizing based on a received pressure signal. As observed in the experiment described above, swapping out the pressure sensor of a vaporizer for MEMS-based thermal flow sensor 114 (i) does not affect the overall airflow versus pressure characteristics of the device, which enables the swap to be carried out without changing the puff resistance that a user may be used to, and (ii) may be carried out without having to change the existing circuitry or processor of the vaporizer, rather the airflow measurement may be simply converted to a pressure measurement.

Reference is now made to FIG. 10, which is a flow chart depicting closed loop sensing for detecting a fouled state of nanoscale resistive element 200, including detecting a severely-fouled state of nanoscale resistive element 200 and cleaning of nanoscale resistive element 200, in accordance with some applications of the present invention. Applying electrical energy to nanoscale resistive element 200 induces a change in an electrical property associated with the application of the electrical energy to nanoscale resistive element 200. For example, the electrical property may be voltage (V), current (I), resistance (R), and/or electrical power (P), as defined by the following equations:

V=IR(Ohm's Law) [Eqn 1]

P=VI=(I\hat{ }2)R=(V\hat{ }2)/R [Eqn 2]

Current flowing through nanoscale resistive element 200 causes an increase in temperature of nanoscale resistive element 200, which in turn causes the resistance of nanoscale resistive element 200 to increase as the electrical energy is applied. The extent of the increase in temperature, however, is affected by how much thermal energy is lost from nanoscale resistive element 200 as the electrical energy is being applied. Thus, the extent of the change in the electrical property is due to the extent of loss of thermal energy from nanoscale resistive element 200 when the electrical energy is applied.

If nanoscale resistive element 200 is clean, and thus surrounded by air, the extent of loss of thermal energy from nanoscale resistive element 200 as the electrical energy is being applied is relatively low (due to air being a good insulator). By contrast, if there is a fouling mass on nanoscale resistive element 200, e.g., some of the vaporizing material, or other particles that may enter flow channel 120, then the extent of loss of thermal energy from nanoscale resistive element 200 as the electrical energy is being applied will be relatively higher (relative to a clean wire) as at least some of the thermal energy is transferred to the fouling mass.

Step 1102 in FIG. 10 represents sensing circuitry 115 operating nanoscale resistive element 200 in a low-power sensing mode, in which sensing circuitry 115 (i) applies electrical energy to nanoscale resistive element 200, e.g., by applying and regulating a voltage across nanoscale resistive element 200, or applying and regulating a current to nanoscale resistive element 200, (ii) detects a change in an electrical property associated with the application of the electrical energy to nanoscale resistive element 200, the extent of the change in the electrical property being due to the extent of loss of thermal energy from nanoscale resistive element 200, and (iii) identifies that nanoscale resistive element 200 is in a fouled state based on the detected change in the electrical property. Thus, a closed loop system is provided, in accordance with some applications of the present invention, in which the sensor is monitored for fouling frequently or generally continuously. Typically, this is a long-term monitoring that filters out changes in the nanoscale resistive element 200 that are due to puffs (sensed as described hereinabove). As indicated by decision diamond 1104, if nanoscale resistive element 200 is not identified to be in a fouled state, then the long-term monitoring continues. For some applications, in response to sensing circuitry 115 determining that nanoscale resistive element 200 is in a fouled state, flow sensor 114 performs the cleaning cycle (step 1110). Typically, during the cleaning cycle, sensing circuitry 115 operates nanoscale resistive element 200 at a burn-off power level that is 50-150 times higher than a sensing power level at which sensing circuitry 115 operates nanoscale resistive element 200 during the low-power sensing mode. For some applications, as indicated by decision diamond 1106, the cleaning cycle is performed only in response to a further determination that nanoscale resistive element 200 is not in a severely-fouled state (further described hereinbelow).

As described hereinabove, current flowing through nanoscale resistive element 200 increases the temperature of nanoscale resistive element 200, thus increasing the resistance of nanoscale resistive element 200. If nanoscale resistive element 200 is in a fouled state then the temperature and the resistance of nanoscale resistive element 200 at any given time while the electrical energy is being applied is lower than the expected temperature and resistance that would occur in the absence of the fouling mass on nanoscale resistive element 200. Thus, for some applications, during the low-power sensing mode, sensing circuitry 115 (i) applies the electrical energy (further described hereinbelow) to nanoscale resistive element 200 to increase the temperature of nanoscale resistive element 200, the increase in temperature of nanoscale resistive element 200 inducing an increase in resistance of nanoscale resistive element 200, (ii) detects the increase in resistance of nanoscale resistive element 200 that is due to the increase in temperature of nanoscale resistive element 200, the extent of the increase in temperature of nanoscale resistive element 200 being due to the extent of loss of thermal energy from nanoscale resistive element 200, and (iii) identifies that nanoscale resistive element 200 is in the fouled state based on the detected increase in resistance of nanoscale resistive element 200.

For some applications, during the low-power sensing mode, sensing circuitry 115 applies the electrical energy to nanoscale resistive element in a CCA mode, in which sensing circuitry 115 regulates a current applied to nanoscale resistive element 200, e.g., sensing circuitry applies a fixed (or otherwise known) current to nanoscale resistive element 200. In this case, sensing circuitry 115 detects the increase in resistance of nanoscale resistive element 200 by monitoring a voltage across nanoscale resistive element 200. The voltage across nanoscale resistive element 200 increases with the increase in resistance of nanoscale resistive element 200 as a fixed current is applied to nanoscale resistive element 200, as per Eqn. 1.

For some applications, during the low-power sensing mode, sensing circuitry 115 applies the electrical energy to nanoscale resistive element in a CVA mode, in which sensing circuitry 115 regulates a voltage applied across nanoscale resistive element 200, e.g., sensing circuitry applies a fixed (or otherwise known) voltage across nanoscale resistive element 200. In this case, sensing circuitry 115 detects the increase in resistance of nanoscale resistive element 200 by monitoring a current in nanoscale resistive element 200 in response to regulating the voltage. The current in nanoscale resistive element 200 decreases with the increase in resistance of nanoscale resistive element 200 as a fixed voltage is applied across nanoscale resistive element 200, as per Eqn. 1.

For some applications, during the low-power sensing mode, sensing circuitry 115 applies the electrical energy (either in CCA mode or CVA mode as described hereinabove) to nanoscale resistive element 200 to increase the temperature of nanoscale resistive element 200 by beginning to apply the electrical energy while nanoscale resistive element 200 is at ambient temperature and the resistance of nanoscale resistive element 200 is at a baseline resistance value, e.g., at least 50 Ohms and/or less than 200 Ohms. The increase in temperature of nanoscale resistive element 200 induces the resistance of nanoscale resistive element 200 to increase to a resistance value that is above the baseline resistance value. Sensing circuitry 115 detects an extent of the increase in the resistance of nanoscale resistive element 200 from the baseline resistance value, and identifies that nanoscale resistive element 200 is in a fouled state in response to the resistance of nanoscale resistive element 200 increasing to a resistance value that is less than a fouled-state threshold value above the baseline resistance value. For some applications, the fouled-state threshold value may be at least 25% and/or less than 75% higher than the baseline resistance value.

For some applications, sensing circuitry 115 determines whether the resistance of nanoscale resistive element 200 has increased to the threshold value above the baseline resistance value at a time t that is while the temperature of nanoscale resistive element 200 is still rising due to the applied electrical energy, e.g., at least 0.1 milliseconds and/or less than 10 milliseconds after the start of the application of the electrical energy to nanoscale resistive element 200.

Furthermore, for some applications, the fouled-state threshold value used in the low-power sensing mode for detecting fouling of nanoscale resistive element 200 is a first threshold value, and sensing circuitry 115 identifies that nanoscale resistive element 200 is in a severely-fouled state in response to the resistance of nanoscale resistive element 200 increasing to a resistance value at time t that is less than a second (severely-fouled-state) threshold value above the baseline resistance value, the second threshold value being lower than the first threshold value. For some applications, the severely-fouled-state threshold value may be at least 10% and/or less than 25% higher than the baseline resistance value. Alternatively or additionally, the severely-fouled-state threshold value is at least 50% and/or less than 99% (e.g., less than 90%) of the first threshold value.

A severely-fouled state may occur, for example, when a droplet of vaporizing material from reservoir 108 entirely encapsulates nanoscale resistive element 200. In the case of severe fouling, cleaning nanoscale resistive element 200 using only the high-power burn-off mode may take longer than a user might want to wait before the vaporizer is ready for use again, e.g., longer than 1 minute. Thus, for some applications, in response to identifying that nanoscale resistive element 200 is in a severely-fouled state, vaporizer 20 generates an alert prompting a user of vaporizer 20 to blow into flow channel 120, as indicated by decision diamond 1106 and step 1112 in FIG. 10. The alert may be a visual alert, e.g., an LED that flashes to indicate to the user that nanoscale resistive element 200 is severely fouled, an audible alert, and/or a tactile alert, e.g., a vibration. Alternatively or additionally, vaporizer 20 may be wirelessly connected to an external device (e.g., via WiFi or BlueTooth), and vaporizer 20 may generate the alert via the external device, e.g., a smartphone application or tablet computer application.

The user's blow through the flow channel may clear the bulk of the contamination off of nanoscale resistive element 200. For some applications, subsequently to the user blowing into flow channel 120, but prior to a new activation of heater 106, sensing circuitry 115 may again operate nanoscale resistive element 200 in the low-power sensing mode, and identify that nanoscale resistive element 200 is in a fouled state in response to the resistance of nanoscale resistive element 200 increasing to a resistance value that is above (I) the second (severely-fouled-state) threshold value above the baseline resistance value and below (II) the first (fouled-state) threshold value above the baseline resistance value. If the measured resistance of nanoscale resistive element 200 is above the second threshold value and below the first threshold value, flow sensor 114 activates the cleaning cycle, i.e., high-power burn-off mode, to clean the remainder of the fouling mass that was not cleared by the user's blow. This provides for a faster way of cleaning a severely-fouled nanoscale resistive element 200 than using the burn-off mode alone. It is noted that it is within the scope of the present invention to use the high-power burn-off mode to clean nanoscale resistive element 200 even when severely-fouled.

For some applications, during the low-power sensing mode, alternatively to applying the electrical energy to nanoscale resistive element 200 in a CCA or CVA mode, sensing circuitry 115 may apply the electrical energy to nanoscale resistive element 200 in a CTA mode, where the temperature of nanoscale resistive element 200 is regulated, e.g., held constant at an elevated temperature above ambient temperature, by varying the electrical power input (e.g., by regulating current in nanoscale resistive element 200 or voltage across nanoscale resistive element 200). Thus, sensing circuitry 115 detects a change in a level of power input to nanoscale resistive element 200 in order to regulate (e.g., hold constant) the temperature of nanoscale resistive element 200, the extent of the change in power input being due to the extent of loss of thermal energy from nanoscale resistive element 200.

Similarly to as described hereinabove with respect to applying the electrical energy in CCA or CVA mode, fouled-state and severely-fouled-state thresholds may be determined for the detected change in the level of power input, mutatis mutandis. At zero electrical power input, nanoscale resistive element 200 is at ambient temperature. According to Eqn. 2, the level of power input will increase in order to maintain nanoscale resistive element at the elevated temperature, and thus at an elevated resistance value. For some applications, a baseline level of power input is considered to be the level of power input at which the elevated temperature of nanoscale resistive element 200 is maintained when nanoscale resistive element 200 is clean. If nanoscale resistive element 200 is fouled and thus loses thermal energy to the fouling mass, then the level of power input to nanoscale resistive element 200 in order to maintain nanoscale resistive element 200 at the elevated temperature will increase to a level of power input that is higher than the baseline level of power input that would occur in the absence of the fouling mass on nanoscale resistive element 200. Thus, sensing circuitry 115 may identify that nanoscale resistive element 200 is in a fouled state in response to the level of power input increasing to a level of power input that is higher than a fouled-state threshold value above the baseline level of power input. Furthermore, for some applications, sensing circuitry 115 may identify that nanoscale resistive element 200 is in a severely-fouled state in response to the level of power input increasing to a level of power input that is higher than a severely-fouled-state threshold value above the baseline level of power input, the severely-fouled-state threshold value being higher than the fouled state threshold value. For some applications, the fouled-state threshold value above the baseline level of power input may be at least 1% (e.g., at least 5%) and/or less than 25% higher than the baseline level of power input. For some applications, the severely-fouled-state threshold value above the baseline level of power input may be at least 25% and/or less than 100% higher than the baseline level of power input. Alternatively or additionally, the severely-fouled-state threshold value is at least 50% and/or less than 99% (e.g., less than 90%) above the fouled-state threshold value.

Reference is now made to FIG. 11, which is a data graph showing a target resistance line 1002 of nanoscale resistive element 200 when nanoscale resistive element 200 is clean and burn-off electrical energy is being applied to nanoscale resistive element 200, and data curves 1004, 1006, and 1008 showing the resistance of nanoscale resistive element 200 during a cleaning cycle, starting from various different degrees of fouling, in accordance with some applications of the present invention. In response to sensing circuitry 115 determining that nanoscale resistive element 200 is in the fouled state (as described hereinabove), flow sensor 114 performs the cleaning cycle in which sensing circuitry 115 operates nanoscale resistive element 200 in a high-power burn-off mode. Typically, during the high-power burn-off mode, sensing circuitry 115 operates nanoscale resistive element 200 at a burn-off power level that is at least 50 and/or less than 150 times higher than a sensing power level at which sensing circuitry 115 operates nanoscale resistive element 200 during the low-power sensing mode. It is noted that even while operating nanoscale resistive element 200 in the low-power sensing mode, the resistance measured as discussed in this paragraph is the resistance of nanoscale resistive element 200 while being heated in response to the sensing voltage applied across nanoscale resistive element 200.

For some applications, during the cleaning cycle, i.e., high-power burn-off mode, sensing circuitry 115 increases the temperature of nanoscale resistive element 200 by applying a burn-off voltage across the nanoscale resistive element in order to vaporize the fouling mass on nanoscale resistive element 200, e.g., by applying a voltage that is at least 1 V and/or less than 2 V, e.g., by applying a voltage that is at least 50 times and/or less than 150 times a sensing voltage applied to nanoscale resistive element 200 during the low-power sensing mode, e.g., in order to measure temperature within the flow channel. For some applications, during burn-off mode, sensing circuitry 115 measures a resistance of nanoscale resistive element 200 in response to the applied burn-off voltage, and may continue operating nanoscale resistive element 200 in burn-off mode for as long as is appropriate to clean nanoscale resistive element 200 by vaporizing all or substantially all of the fouling mass, e.g., sensing circuitry 115 terminates the cleaning cycle when the resistance of nanoscale resistive element 200 passes a clean-state threshold value.

For some applications, sensing circuitry 115 may measure the resistance of nanoscale resistive element 200 in response to the applied burn-off voltage during operation of nanoscale resistive element 200 in the high-power burn-off mode. Flow sensor 114 may terminate the cleaning cycle when the resistance of nanoscale resistive element 200 passes a clean-state threshold value in response to the applied burn-off voltage. It is noted that while FIG. 11 depicts the data as resistance versus time, the same data could be plotted as temperature of nanoscale resistive element 200 versus time.

Target resistance line 1002 in FIG. 11 represents the resistance of nanoscale resistive element 200 in response to the applied electrical energy when nanoscale resistive element 200 is in a clean state. Data curves 1004, 1006, and 1008 represent three examples of the resistance of a fouled nanoscale resistive element 200 in response to the applied burn-off voltage during operation of nanoscale resistive element 200 in burn-off mode. Each of the three examples starts with a different degree of fouling, due to successively greater amounts of time in which the corresponding nanoscale resistive element 200 was pre-exposed to fouling. Curve 1004 represents 5 seconds of pre-exposure to fouling, curve 1006 represents 25 seconds of pre-exposure to fouling, and curve 1008 represents 50 seconds of pre-exposure to fouling.
Curve 1004 represents the least fouled starting point out of the three examples, and as nanoscale resistive element 200 is operated in burn-off mode, the resistance of nanoscale resistive element 200 in this example increases to the target resistance value relatively quickly, after about 500 ms.
Curve 1006 represents a nanoscale resistive element 200 with a higher degree of fouling. In this example, although it takes longer (about 2500 ms), the resistance of nanoscale resistive element 200 increases to the target resistance value in response to being operated in burn-off mode.
Curve 1008 represents a very fouled nanoscale resistive element 200. Although taking even longer than curve 1006, the resistance of nanoscale resistive element 200 in this example does eventually increase to the target resistance value, after about 4500 ms.
   The inventors hypothesize based on this data that any reasonable amount of fouling mass on nanoscale resistive element 200 may be vaporized and nanoscale resistive element 200 brought to the target resistance value, provided nanoscale resistive element 200 is maintained in burn-off mode for long enough.

Applications of the invention described herein can take the form of a computer program product accessible from a computer-usable or computer-readable medium (e.g., a non-transitory computer-readable medium) providing program code for use by or in connection with a computer or any instruction execution system, such as microcontroller 404. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD. For some applications, cloud storage, and/or storage in a remote server is used.

A data processing system suitable for storing and/or executing program code will include at least one processor (e.g., microcontroller 404) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the methods described herein can be implemented by computer program instructions. These computer program instructions may be provided to a processor of a general-purpose computer, special purpose computer, or other programmable data processing apparatus to produce a machine, such that the instructions, which execute via the processor of the computer (e.g., microcontroller 404) or other programmable data processing apparatus, create means for implementing the functions/acts specified in the methods described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the methods described in the present application. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the methods described in the present application.

Microcontroller 404 and the other computer processors described herein are typically hardware devices programmed with computer program instructions to produce a special purpose computer. For example, when programmed to perform the methods described herein, the computer processor typically acts as a special purpose computer processor. Typically, the operations described herein that are performed by computer processors transform the physical state of a memory, which is a real physical article, to have a different magnetic polarity, electrical charge, or the like depending on the technology of the memory that is used.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising:
a vaporizer shaped to define a flow channel that is open to an environment external to the vaporizer at first and second ends of the flow channel, the first end of the flow channel being at a mouthpiece of the vaporizer; and
a flow sensor comprising (a) a nanoscale resistive element disposed at least partially within the flow channel and (b) sensing circuitry configured to measure a change in the nanoscale resistive element due to airflow within the flow channel.

2-3. (canceled)

4. The apparatus according to claim 1, wherein the flow sensor comprises a flow sensor housing, (a) the nanoscale resistive element and the sensing circuitry being disposed within the flow sensor housing, and (b) the flow sensor housing being disposed at least partially within the flow channel.

5-9. (canceled)

10. The apparatus according to claim 1, wherein the vaporizer comprises a reservoir configured to hold a material for being vaporized, and wherein the nanoscale resistive element is disposed 5-100 mm away from the reservoir.

11. (canceled)

12. The apparatus according to claim 1, wherein:
(a) the vaporizer comprises a heater for vaporizing a material within the vaporizer,
(b) the sensing circuitry is configured to measure a velocity of the airflow within the flow channel in response to a measured change in the nanoscale resistive element, and
(c) the vaporizer is configured to activate the heater when the measured velocity of the airflow within the flow channel reaches a threshold value.

13. The apparatus according to claim 12, wherein:
(a) the sensing circuitry comprises switching circuitry, configured to switch the sensing circuitry between:
(i) measuring velocity of the airflow within the flow channel in response to a measured change in the nanoscale resistive element, and
(ii) measuring temperature within the flow channel in response to a measured change in the nanoscale resistive element,
(b) the flow sensor is configured to determine a temperature-compensated velocity value of the airflow within the flow channel by using the measured temperature to correct the measured velocity of the airflow, and
(c) the vaporizer is configured to activate the heater when the temperature-compensated velocity value reaches a threshold value.

14. The apparatus according to claim 13, wherein the switching circuitry is configured to switch the sensing circuitry between (i) operating the nanoscale resistive element in a Constant Temperature Anemometry (CTA) mode of operation, in which the sensing circuitry measures velocity of the airflow within the flow channel in response to a measured change in the nanoscale resistive element, and (ii) operating the nanoscale resistive element in a Constant Current Anemometry (CCA) mode of operation, in which the sensing circuitry measures temperature within the flow channel in response to a measured change in the nanoscale resistive element.

15-16. (canceled)

17. The apparatus according to claim 13, wherein the sensing circuitry is configured to operate the nanoscale resistive element in a Constant Voltage Anemometry (CVA) mode of operation, and wherein the switching circuitry is configured to switch the sensing circuitry between (i) operating the nanoscale resistive element at a first voltage level such that the sensing circuitry measures velocity of the airflow within the flow channel in response to a measured change in the nanoscale resistive element and (ii) operating the nanoscale resistive element at a second voltage level that is lower than the first voltage level such that the sensing circuitry measures temperature within the flow channel in response to a measured change in the nanoscale resistive element.

18. The apparatus according to claim 12, wherein:
(a) the nanoscale resistive element is a first nanoscale resistive element,
(b) the flow sensor further comprises a second nanoscale resistive element,
(c) the sensing circuitry is configured to operate the first and second nanoscale resistive elements, such that:
(i) the sensing circuitry measures velocity of the airflow within the flow channel in response to a measured change in the first nanoscale resistive element, and
(ii) the sensing circuitry measures temperature within the flow channel in response to a measured change in the second nanoscale resistive element,
(d) the flow sensor is configured to determine a temperature-compensated velocity value of the airflow within the flow channel by using the measured temperature to correct the measured velocity of the airflow, and
(e) the vaporizer is configured to activate the heater when the temperature-compensated velocity value reaches a threshold value.

19-23. (canceled)

24. The apparatus according to claim 12, wherein the flow sensor is configured to perform a cleaning cycle, the sensing circuitry being configured to increase the temperature of the nanoscale resistive element during the cleaning cycle, and wherein the sensing circuitry is configured to increase the temperature of the nanoscale resistive element to 300-1000 degrees Celsius during the cleaning cycle.

25-26. (canceled)

27. The apparatus according to claim 12, wherein the flow sensor is configured to perform a cleaning cycle, the sensing circuitry being configured to increase the temperature of the nanoscale resistive element during the cleaning cycle, and, wherein:
(A) the sensing circuitry is configured to operate the nanoscale resistive element in a low-power sensing mode in which the sensing circuitry is configured to:
(i) apply electrical energy to the nanoscale resistive element,
(ii) detect a change in an electrical property associated with the application of the electrical energy to the nanoscale resistive element, the extent of the change in the electrical property being due to the extent of loss of thermal energy from the nanoscale resistive element, and
(iii) identify that the nanoscale resistive element is in a fouled state based on the detected change in the electrical property, and
(B) the flow sensor is configured to perform the cleaning cycle in response to the sensing circuitry determining that the nanoscale resistive element is in the fouled state.

28. (canceled)

29. The apparatus according to claim 27, wherein the sensing circuitry is configured to, during the low-power sensing mode:
(i) apply the electrical energy to the nanoscale resistive element to increase the temperature of the nanoscale resistive element, the increase in temperature of the nanoscale resistive element inducing an increase in resistance of the nanoscale resistive element,
(ii) detect the increase in resistance of the nanoscale resistive element that is due to the increase in temperature of the nanoscale resistive element, the extent of the increase in temperature of the nanoscale resistive element being due to the extent of loss of thermal energy from the nanoscale resistive element, and
(iii) identify that the nanoscale resistive element is in the fouled state based on the detected increase in resistance of the nanoscale resistive element.

30-31. (canceled)

32. The apparatus according to claim 29, wherein the sensing circuitry is configured to, during the low-power sensing mode:
(i) apply the electrical energy to the nanoscale resistive element to increase the temperature of the nanoscale resistive element by beginning to apply the electrical energy while the resistance of the nanoscale resistive element is at a baseline resistance value, the increase in temperature of the nanoscale resistive element inducing the resistance of the nanoscale resistive element to increase to a resistance value that is above the baseline resistance value,
(ii) detect an extent of the increase in the resistance of the nanoscale resistive element from the baseline resistance value, and
(iii) identify that the nanoscale resistive element is in the fouled state in response to the resistance of the nanoscale resistive element increasing to a resistance value that is less than a threshold value above the baseline resistance value.

33-39. (canceled)

40. The apparatus according to claim 27, wherein the sensing circuitry is configured to, during the low-power sensing mode:
(i) regulate the temperature of the nanoscale resistive element by applying the electrical energy to the nanoscale resistive element,
(ii) detect a change in a level of power input to the nanoscale resistive element in order to regulate the temperature of the nanoscale resistive element, the extent of the change in power input being due to the extent of loss of thermal energy from the nanoscale resistive element, and
(iii) identify that the nanoscale resistive element is in the fouled state based on the change in the level of power input to the nanoscale resistive element.

41-43. (canceled)

44. The apparatus according to claim 12, wherein the flow sensor is configured to perform a cleaning cycle, the sensing circuitry being configured to increase the temperature of the nanoscale resistive element during the cleaning cycle, and, wherein:
(A) during the cleaning cycle, the sensing circuitry is configured to:
(i) increase the temperature of the nanoscale resistive element by applying a voltage across the nanoscale resistive element, and
(ii) monitor the temperature of the nanoscale resistive element in response to the applied voltage, and
(B) the flow sensor is configured to terminate the cleaning cycle when the temperature of the nanoscale resistive element passes a clean-state threshold value.

45-46. (canceled)

47. The apparatus according to claim 12, wherein the flow sensor is configured to determine an amount of material vaporized within the vaporizer in response to the measured velocity of the airflow within the flow channel subsequent to the activation of the heater.

48-54. (canceled)

55. The apparatus according to claim 12, wherein the sensing circuitry is configured to:
(a) operate the nanoscale resistive element in a low-power Constant Temperature Anemometry (CTA) puff detection mode, in which the nanoscale resistive element is maintained at a constant differential temperature relative to ambient temperature, and
(b) when a puff is detected, switch to operating the nanoscale resistive element in a high-power CTA puff characterization mode, in which the nanoscale resistive element is maintained at a constant absolute temperature.

56. The apparatus according to claim 12, wherein the sensing circuitry is configured to:
(a) operate the nanoscale resistive element in a low-power Constant Temperature Anemometry (CTA) puff detection mode, in which the sensing circuitry intermittently heats the nanoscale resistive element in order to measure the velocity of the airflow within the flow channel, and
(b) when a puff is detected, switch to a high-power CTA puff characterization mode, in which the nanoscale resistive element is maintained at a constant absolute temperature.

57-58. (canceled)

59. The apparatus according to claim 12, wherein the flow sensor is configured to determine a direction of the airflow within the flow channel in response to the sensing circuitry measuring a change in temperature of the nanoscale resistive element due to airflow within the flow channel.

60. The apparatus according to claim 12, wherein:
the sensing circuitry is configured to generate a sensor signal indicative of the measured velocity of the airflow within the flow channel,
the vaporizer comprises a processor configured to analyze a level of fluctuations in the sensor signal, and
the processor is configured to determine a direction of the airflow within the flow channel in response to the level of fluctuations in the sensor signal.

61-76. (canceled)

77. A method for measuring airflow inside a vaporizer, the method comprising:
using a flow sensor disposed within the flow channel of a vaporizer, (i) the vaporizer being shaped to define a flow channel that is open to an environment external to the vaporizer at first and second ends of the flow channel, the first flow channel being at a mouthpiece of the vaporizer, and (ii) the flow sensor comprising at least one nanoscale resistive element disposed at least partially within the flow channel:
measuring a velocity of the airflow within the flow channel by measuring a change in the at least one nanoscale resistive element due to airflow within the flow channel; and
activating a heater for vaporizing a material within the vaporizer when the measured velocity reaches a threshold value,
the method further comprising:
measuring a temperature within the flow channel by measuring a change in a nanoscale resistive element selected from the group consisting of: the at least one nanoscale resistive element, and another nanoscale resistive element;
determining a temperature-compensated velocity value by using the measurement of the temperature within the flow channel to correct the measured velocity of the airflow; and
activating the heater when the temperature-compensated velocity value reaches a threshold value.

78. (canceled)
